(19) United States
(12) Patent Application Publication
(10) Pub. No.: US 2017/0231832 A1
(43) Pub. Date: Aug. 17, 2017

(54) APPARATUS AND METHOD OF COMPRESSION

(71) Applicant: Kimberly-Clark Worldwide, Inc, Neenah, WI (US)

(72) Inventors: Ronald Alex HILT, Oshkosh, WI (US); Steven Craig GEHLING, Oshkosh, WI (US)

(21) Appl. No.: 15/514,705

(22) PCT Filed: Sep. 30, 2014
(86) PCT No.: PCT/US2014/058207

§ 371 Date: Mar. 27, 2017

(51) Int. Cl. A61F 13/20; A61F 2/00

(52) U.S. Cl. CPC A61F 13/2088; A61F 13/2054; A61F 2/0004; A61F 13/2034

(57) ABSTRACT

An apparatus for compressing a material such as a tampon or a pessary device, and a method of compressing a material such as a tampon or a pessary device are described. The apparatus can have a first press unit support structure and a second press unit support structure, and each of the first press unit support structure and the second press unit support structure is rotatable about an axis. A first press unit has a compression surface area which decreases with the movement of compression associated with the first press unit support structure. A second press unit is associated with the second press unit support structure, wherein the second press unit is one of an axial direction press unit, a non-linear direction press unit, or a press unit having a compression surface area which decreases with the movement of compression.

### BACKGROUND

A wide variety of products can undergo a compression step during a manufacturing process of the product. Compression of the product can alter the dimensions of the product from its original starting dimensions and reduce those dimensions to render a product with final smaller dimensions. Examples of personal care products which can undergo a compression step in a manufacturing process can include tampons and pessaries.

Tampons and pessaries generally undergo a compression step during the manufacturing process in order to render the product into a size and dimension more suitable for insertion into the body of the user. The compression of a tampon pledget or uncompressed pessary can result in a tampon or compressed pessary capable of being inserted digitally by the user's fingers or through the use of an applicator. A tampon is generally manufactured by folding, rolling, or stacking an absorbent structure made of loosely associated absorbent material into a pledget. The pledget can then be compressed into a tampon of the desired size and shape. A pessary can similarly be manufactured from an absorbent material, or can be manufactured from non-absorbent material, and can ultimately be compressed into a size suitable for insertion into the vaginal cavity.

Current manufacturing processes generally compress pledgets or pessaries one at a time. An apparatus which can compress only one tampon pledget or pessary at a time can result in limitations in the production efficiency of finished tampons and pessaries. A limitation can be the decrease in productive time and an increase in the non-productive time during the compression step of a manufacturing process. Productive time, for example, can be the time during which the pledget or uncompressed pessary is being transformed into a final tampon or compressed pessary. Non-productive time, for example, can be the time during which the pledget or uncompressed pessary is waiting for an action to be taken upon itself, such as, for example, time spent waiting for the pledget or uncompressed pessary to enter the compression apparatus. Another example of a limitation can be the volume of synchronous operations versus asynchronous operations. During synchronous operations, productive and non-productive operations can occur simultaneously with one or more other productive or non-productive operations. During asynchronous operations, productive and non-productive operations can occur sequentially with other productive or non-productive operations. A larger volume of asynchronous operations, particularly non-productive asynchronous operations can decrease the efficiency of the production of tampons and pessaries.

One attempt to address these limitations related to the compression step of manufacturing processes has been to speed up the revolution time of the compression apparatus. Increasing the revolution time of the apparatus, however, has failed to change the overall efficiency of the apparatus as only one pledget or pessary is being compressed within the single revolution of the compression apparatus. There is a need for an apparatus which can compress more than a single tampon pledget or pessary in one revolution of the apparatus.

### SUMMARY

In various embodiments, an apparatus can have a first press unit support structure and a second press unit support structure, each of the first press unit support structure and the second press unit support structure rotatable about an axis; an axial direction press unit associated with the first press unit support structure; and a second press unit associated with the second press unit support structure wherein the second press unit is one of an axial direction press unit, a non-linear direction press unit, or a press unit having a compression surface area which decreases over time. In various embodiments, at a moment in time during a revolution of each of the first and second press unit support structures about the axis, the axial direction press unit is in a configuration which is one of a full open configuration, partially closed configuration, a partially open configuration, or a full closed configuration and the second press unit is in a configuration which is one of a full open configuration, a partially closed configuration, a partially open configuration, or a full closed configuration. In various embodiments, the configuration of the axial direction press unit is the same as the configuration of the second press unit. In various embodiments, the configuration of the axial direction press unit is different from the configuration of the second press unit. In various embodiments, the axial direction press unit is associated with the first press unit support structure in a variable spatial relationship relative to second press unit which is associated with the second press unit support structure. In various embodiments, compression of a material within one of the axial press unit or second press unit begins after the axial press unit or second press unit rotates from a zero degree position and continues to a rotation of at least about a 90 degree position.

In various embodiments, an apparatus can have a first press unit support structure and a second press unit support structure, each of the first press unit support structure and the second press unit support structure rotatable about an axis; a non-linear direction press unit associated with the first press unit support structure; and a second press unit associated with the second press unit support structure wherein the press unit is one of an axial direction press unit, a non-linear direction press unit, or a press unit having a compression surface area which decreases with the movement of compression. In various embodiments, at a moment in time during a revolution of each of the first and second press unit support structures about the axis, the non-linear direction press unit is in a configuration which is one of a full open configuration, a partially closed configuration, a full closed configuration or a partially open configuration and the second press unit is in a configuration which is one of a full open configuration, a partially closed configuration, a full closed configuration or a partially open configuration. In various embodiments, the configuration of the non-linear direction press unit is the same as the configuration of the second press unit. In various embodiments, the configuration of the non-linear direction press unit is different from the configuration of the second press unit. In various embodiments, the non-linear direction press unit is associated with the first press unit support structure in a variable spatial relationship relative to second press unit which is associated with the second press unit support structure. In various embodiments, compression of a material within one of the non-linear direction press unit or the second press unit begins after the non-linear direction press unit or the second press unit rotates from a zero degree position and continues to a rotation of at least about a 90 degree position.

In various embodiments, an apparatus can have a first press unit support structure and a second press unit support structure, each of the first press unit support structure and the second press unit support structure rotatable about an axis; a first press unit having a compression surface area which decreases with the movement of compression associated with the first press unit support structure; and a second press unit associated with the second press unit support structure, wherein the second press unit is one of an axial direction press unit, a non-linear direction press unit, or a press unit having a compression surface area which decreases with the movement of compression. In various embodiments, at a moment in time during a revolution of each of the first and second press unit support structures about the axis, the first press unit is in a configuration which is one of a full open configuration, a partially closed configuration, a partially open configuration, or a full closed configuration and the second press unit is in a phase which is one of a full open configuration, a partially closed configuration, a partially open configuration or a full closed configuration. In various embodiments, the configuration of the first press unit is the same as the configuration of the second press unit. In various embodiments, the configuration of the first press unit is different from the configuration of the second press unit. In various embodiments, the first press unit is associated with the first press unit support structure in a variable spatial relationship relative to the second press unit which is associated with the second press unit support structure. In various embodiments, compression of a material within one of the first or second press units begins after the first or second press units rotates from a zero degree position and continues to a rotation of at least about a 90 degree position.

### BRIEF DESCRIPTION OF THE DRAWINGS

FIG. 1A is a perspective view of an exemplary embodiment of an absorbent structure.

FIG. 1B is a top down view of an exemplary embodiment of an absorbent structure.

FIGS. 2A and 2B are perspective views of exemplary embodiments of pledgets.

FIGS. 3A-3D are side views of exemplary embodiments of tampons.

FIG. 4A is a perspective view of an exemplary embodiment of a pessary.

FIG. 4B is a perspective view of an exemplary embodiment of a core of the pessary of FIG. 4A.

FIG. 4C is a perspective view of an exemplary embodiment of the compressed core of the pessary of FIG. 4A.

FIG. 5A is a perspective view of an exemplary embodiment of a pessary with a fold.

FIG. 5B is a cross-sectional view of the pessary of FIG. 5A.

FIG. 6A is a perspective view of an exemplary embodiment of a pessary with a strut.

FIG. 6B is a cross-sectional view of the pessary of FIG. 6A.

FIG. 7 is a schematic view of an exemplary embodiment of an apparatus.

FIGS. 8A-8E are schematic illustrations of an exemplary embodiment of axial compression in the longitudinal direction.

FIGS. 9A-9C are schematic illustrations of an exemplary embodiment of axial compression in the lateral direction.

FIG. 10 is an exemplary embodiment of a non-linear direction press unit.

FIG. 11A is an exemplary embodiment of the press unit of FIG. 10 in an open phase.

FIG. 11B is an exemplary embodiment of the press unit of FIG. 10 in a partially closed phase.

FIG. 11C is an exemplary embodiment of the press unit of FIG. 10 in a closed phase.

FIG. 12 is a schematic illustration of an exemplary embodiment of a non-linear direction press unit in an open phase.

FIG. 13 is a schematic illustration of an exemplary embodiment of a non-linear direction press unit in a closed phase.

FIG. 14 illustrates a broad side view of an exemplary indentation press jaw.

FIG. 14A illustrates an enlarged view of detail A of FIG. 14.

FIG. 15 illustrates a broad side view of an exemplary indentation press jaw.

FIG. 15A illustrates an enlarged view of detail A of FIG. 15.

FIG. 16 illustrates a broad side view of an exemplary indentation press jaw.

FIG. 16A illustrates an enlarged view of detail A of FIG. 16.

FIG. 17 illustrates a broad side view of an exemplary indentation press jaw.

FIGS. 17A and 17B illustrate enlarged views of details A and B, respectively, of FIG. 17.

FIG. 18 illustrates a broad side view of an exemplary indentation press jaw.

FIG. 18A illustrates an enlarged view of detail A of FIG. 18.

FIG. 19 is a schematic illustration of an exemplary embodiment of a press unit having a compression surface area which decreases during compression in an open phase.

FIG. 20 is a schematic illustration of an exemplary embodiment of a press unit having a compression surface area which decreases during compression in a closed phase.

FIG. 21 illustrates a lever and jaw used in the press unit of FIG. 19 and FIG. 20.

### DETAILED DESCRIPTION

The present disclosure is generally directed towards an apparatus which can be used in the compression step of a manufacturing process of a tampon or pessary. The present disclosure is also generally directed towards a process of compressing a material, such as, for example, a pledget or a pessary.

### Definitions

The term “applicator” refers herein to a device that facilitates the insertion of a tampon or pessary into the vaginal cavity of a female. Non-limiting examples of such include any known hygienically designed applicator that is capable of receiving a tampon or a pessary, including the so-called telescoping, barrel and plunger, and compact applicators.

The term “attached” refers herein to configurations in which a first element is secured to a second element by joining the first element to the second element. Joining the first element to the second element can occur by joining the first element directly to the second element, indirectly such as by joining the first element to an intermediate member(s) which in turn can be joined to the second element, and in configurations in which the first element is integral with the second element (i.e., the first element is essentially part of the second element). Attachment can occur by any method deemed suitable including, but not limited to, adhesives, ultrasonic bonds, thermal bonds, pressure bonds, mechanical entanglement, hydroentanglement, microwave bonds, or any other conventional technique. The attachment can extend continuously along the length of attachment, or it may be applied in an intermittent fashion at discrete intervals.

The term “bicomponent fiber” refers herein to fibers that have been formed from at least two different polymers extruded from separate extruders but spun together to form one fiber. Bicomponent fibers are also sometimes referred to as conjugate fibers or multicomponent fibers. The polymers can be arranged in substantially constantly positioned distinct zones across the cross-section of the bicomponent fiber and can extend continuously along the length of the bicomponent fiber. The configuration of such a bicomponent fiber may be, for example, a sheath/core arrangement wherein one polymer is surrounded by another or may be a side-by-side arrangement, a pie arrangement, or an “islands-in-the-sea” arrangement.

The term “compression” refers herein to the process of pressing, squeezing, compacting, or otherwise manipulating the size, shape, and/or volume of a material to obtain an insertable tampon or pessary. For example, a pledget can undergo compression to obtain a tampon having a vaginally insertable shape. The term “compressed” refers herein to the state of the material(s) subsequent to compression. Conversely, the term “uncompressed” refers herein to the state of the material(s) prior to compression. The term “compressible” is the ability of the material to undergo compression.

The term “cross-section” refers herein to a plane of the tampon or pessary that extends laterally through the tampon or pessary and which is orthogonal to the longitudinal axis of the tampon or pessary or which is transverse or perpendicular to the longitudinal axis.

The term “digital tampon” refers herein to a tampon, which is intended to be inserted into the vaginal cavity with the user's finger and without the aid of an applicator. Thus, digital tampons are typically visible to the user prior to use rather than being housed in an applicator.

The term “folded” refers herein to the configuration of a pledget that can be incidental to lateral compaction of the absorbent structure of the pledget or may purposefully occur prior to a compression step. Such a configuration can be readily recognizable, for example, when the absorbent material of the absorbent structure abruptly changes direction such that one part of the absorbent structure bends or lies over another part of the absorbent structure.

The term “generally cylindrical” refers herein to the usual shape of tampons as is well known in the art, but which also includes oblate or partially flattened cylinders, curved cylinders, and shapes which have varying cross-sectional areas (e.g., bottle shaped) along the longitudinal axis.

The term “longitudinal axis” refers herein to the axis running in the direction of the longest linear dimension of the tampon or pessary. For example, the longitudinal axis of a tampon is the axis which runs from the insertion end to the withdrawal end. As another example, the longitudinal axis of a pessary is the axis which runs from the anchoring element to the supporting element.

The term “outer surface” refers herein to the visible surface of the (compressed and/or shaped) tampon or pessary prior to use and/or expansion. As least part of the outer surface may be smooth or alternatively may have topographical features, such as ribs, spiraling ribs, grooves, a mesh pattern or other topographical features.

The term “pessary” refers herein to a device used to treat urinary incontinence. A pessary can have an anchoring element, a supporting element, and a withdrawal element.

The term “pledget” refers herein to a construction of an absorbent structure prior to the compression and/or shaping of the absorbent structure into a tampon. The absorbent structure may be rolled, folded, or otherwise manipulated into a pledget prior to compression of the pledget. Pledgets are sometimes referred to as blanks or softwinds, and the term “pledget” is intended to include such terms as well. In general, the term “tampon” is used to refer to a finished tampon after the compression and/or shaping process.

The term “radial axis” refers herein to the axis that runs at right angles to the longitudinal axis of the tampon or pessary.

The term “relatively smooth” refers herein to a surface relatively free from irregularities, roughness, or projections greater than about 1 mm in height or depth as measured from the surface.

The term “rolled” refers herein to a configuration of the pledget after winding the absorbent structure upon itself.

The term “tampon” refers herein to an absorbent structure that is inserted into the vaginal cavity for the absorption of fluid therefrom or for the delivery of active materials, such as medicaments. A pledget may have been compressed in the non-linear direction, an axial direction along the longitudinal and/or lateral axis, or in both the non-linear and axial directions to form a generally cylindrical tampon. While the tampon can be in a substantially cylindrical configuration, other shapes are possible. These other shapes can include, but are not limited to, having a cross-section that can be described as rectangular, triangular, trapezoidal, semi-circular, hourglass, serpentine, or other suitable shapes. Tampons have an insertion end, a withdrawal end, a withdrawal element, a length, a width, a longitudinal axis, a radial axis, and an outer surface. The tampon's length can be measured from the insertion end to the withdrawal end along the longitudinal axis. A typical tampon can have a length from about 30 mm to about 60 mm. A tampon can be linear or non-linear in shape, such as curved along the longitudinal axis. A typical tampon can have a width from about 2 mm to about 30 mm. The width of the tampon, unless otherwise stated, corresponds to the length across the largest transverse cross-section, along the length of the tampon.

The term “vaginal cavity” refers herein to the internal genitalia of the mammalian female in the pudendal region of the body. The term generally refers to the space located between the introitus of the vagina (sometimes referred to as the sphincter of the vagina or the hymeneal ring) and the cervix. The term does not include the interlabial space, the floor of the vestibule or the externally visible genitalia.

As noted above, personal care products which can undergo a compression step during the manufacturing process can include, but are not limited to, tampons and pessaries.

### Tampon:

A tampon can result from the compression of a pledget. The pledget, in turn, can be formed from an absorbent structure composed of absorbent material.

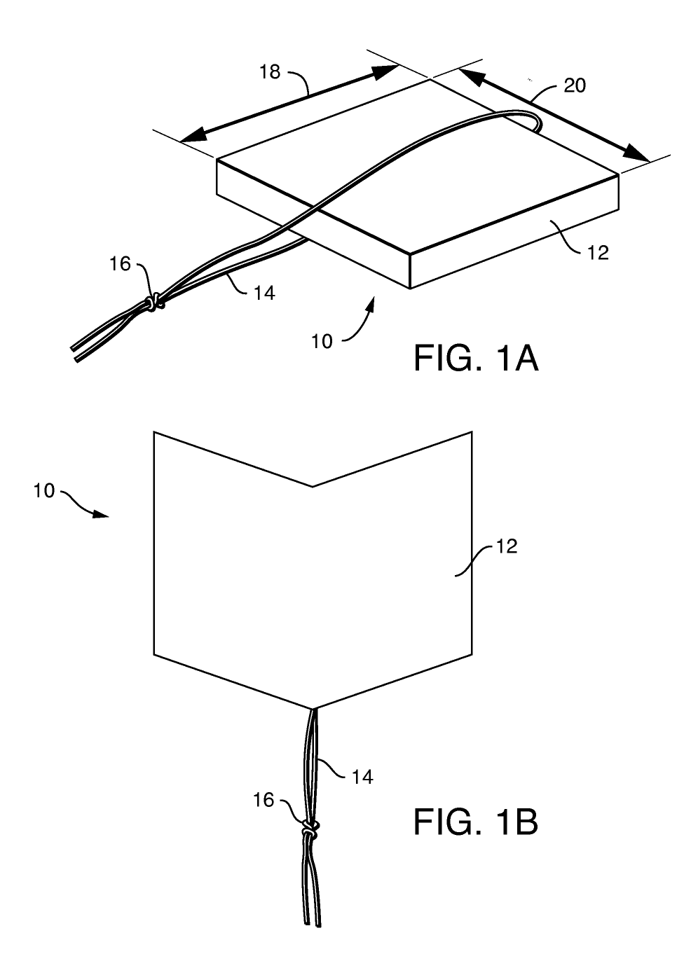

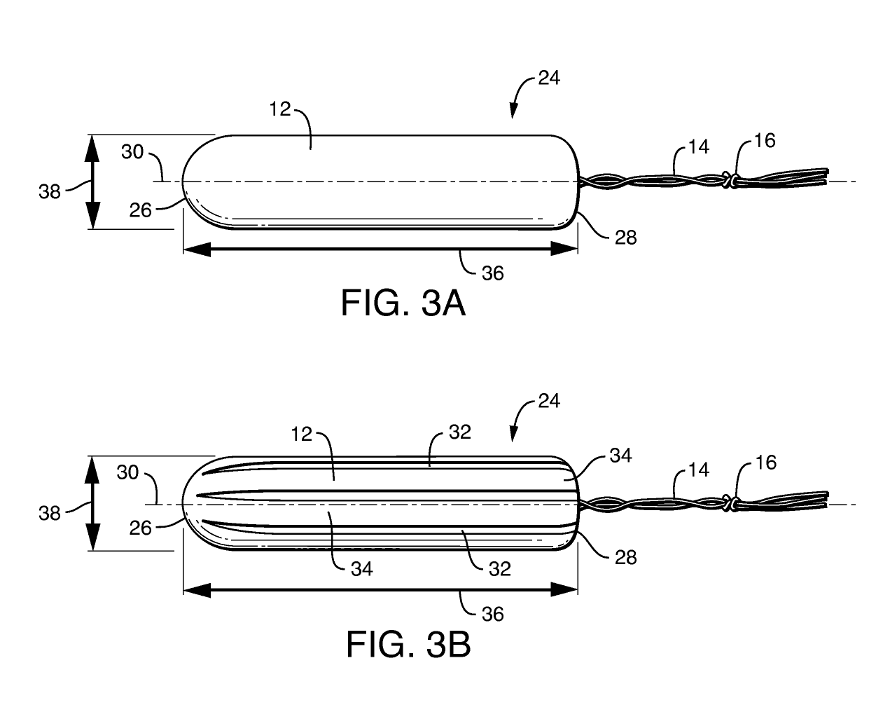

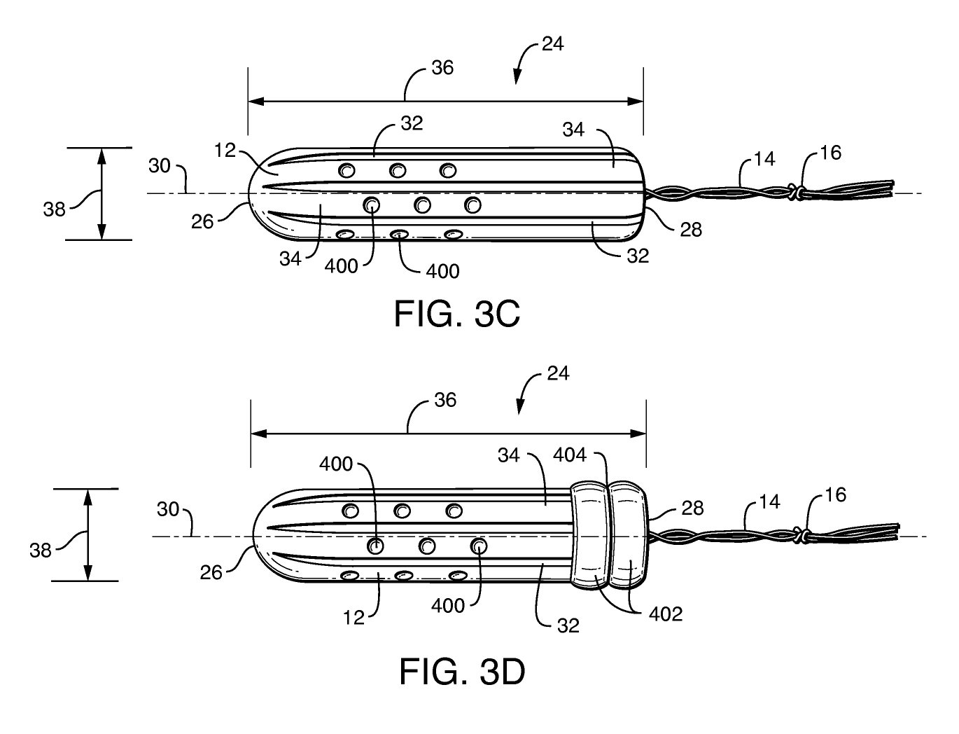

FIG. 1A illustrates a perspective view of an exemplary embodiment of an absorbent structure 10 generally in the shape of a square and a withdrawal element 14 having a knot 16 associated with the absorbent structure 10. FIG. 1B illustrates a top down view of an exemplary embodiment of an absorbent structure 10 having a generally chevron shape and a withdrawal element 14 having a knot 16 associated with the absorbent structure 10. It is to be understood that these two shapes, square and chevron, are illustrative and the absorbent structure 10 can have any shape, size and thickness that can ultimately be compressed into a tampon, such as, for example, tampon 24 in FIGS. 3A-3D. Non-limiting examples of the shape of an absorbent structure 10 can include, but are not limited to, oval, round, chevron, square, rectangular, and the like. The absorbent structure 10 can have a single layer of absorbent material 12 or the absorbent structure 10 can be a laminar structure that can have individual distinct layers of absorbent material 12. In an embodiment in which the absorbent structure 10 has a laminar structure, the layers can be formed from a single absorbent material and/or from different absorbent materials. In an embodiment, the absorbent structure 10 can have a length dimension 18 along the longitudinal axis of the absorbent structure 10 from about 20, 30 or 40 mm to about 50, 60, 75, 100, 200, 250 or 300 mm. In an embodiment, the absorbent structure 10 can have a width dimension 20 lateral to the longitudinal axis of the absorbent structure 10 from about 40 mm to about 80 mm. In an embodiment, the basis weight of the absorbent structure 10 can range from about 15, 20, 25, 50, 75, 90, 100, 110, 120, 135, or 150 gsm to about 1,000, 1,100, 1,200, 1,300, 1,400, or 1,500 gsm.

The absorbent material 12 of the absorbent structure 10 can be absorbent fibrous material. Such absorbent material 12 can include, but is not limited to, natural and synthetic fibers such as, but not limited to, polyester, acetate, nylon, cellulosic fibers such as wood pulp, cotton, rayon, viscose, LYOCELL® such as from Lenzing Company of Austria, or mixtures of these or other cellulosic fibers. Natural fibers can include, but are not limited to, wool, cotton, flax, hemp, and wood pulp. Wood pulps can include, but are not limited to, standard softwood fluffing grade such as CR-1654 (US Alliance Pulp Mills, Coosa, Ala.). Pulp may be modified in order to enhance the inherent characteristics of the fibers and their processability, such as, for example, by crimping, curling, and/or stiffening. The absorbent material 12 can include any suitable blend of fibers.

In an embodiment, the absorbent structure 10 can contain fibers such as binder fibers. In an embodiment, the binder fibers can have a fiber component which will bond or fuse to other fibers in the absorbent structure 10. Binder fibers can be natural fibers or synthetic fibers. Synthetic fibers include, but are not limited to, those made from polyolefins, polyamides, polyesters, rayon, acrylics, viscose, superabsorbents, LYOCELL® regenerated cellulose and any other suitable synthetic fiber known to those skilled in the art. The fibers can be treated by conventional compositions and/or processes to enable or enhance wettability.

In various embodiments, the absorbent structure 10 can have any suitable combination and ratio of fibers. In an embodiment, the absorbent structure 10 can include from about 70 to about 95 wt % absorbent fibers and from about 5 to about 30 wt % binder fibers.

In various embodiments, a cover can be provided as known to one of ordinary skill in the art. As used herein, the term “cover” relates to materials that are in communication with and cover or enclose surfaces, such as, for example, an outer surface of the tampon 24 and reduce the ability of portions (e.g., fibers and the like) from becoming separated from the tampon 24 and being left behind upon removal of the tampon 24 from the woman's vaginal cavity.

In various embodiments, the cover can be formed from nonwoven materials or apertured films. The cover can be made by any number of suitable techniques such as, for example, being spunbond, carded, hydroentangled, thermally bonded, and resin bonded. In an embodiment, the cover can be a 12 gsm smooth calendared material made from bicomponent, polyester sheath and polyethylene core, fibers such as Sawabond 4189 available from Sandler AG, Schwarzenbach, Germany.

In various embodiments, the absorbent structure 10 may be attached to a withdrawal element 14. The withdrawal element 14 may be attached to the absorbent structure 10 in any suitable manner as known to one of ordinary skill in the art. A knot 16 can be formed near the free ends of the withdrawal element 14 to assure that the withdrawal element 14 does not separate from the absorbent structure 10. The knot 16 can also serve to prevent fraying of the withdrawal element 14 and to provide a place or point where a woman can grasp the withdrawal element 14 when she is ready to remove the tampon 24 from her vaginal cavity.

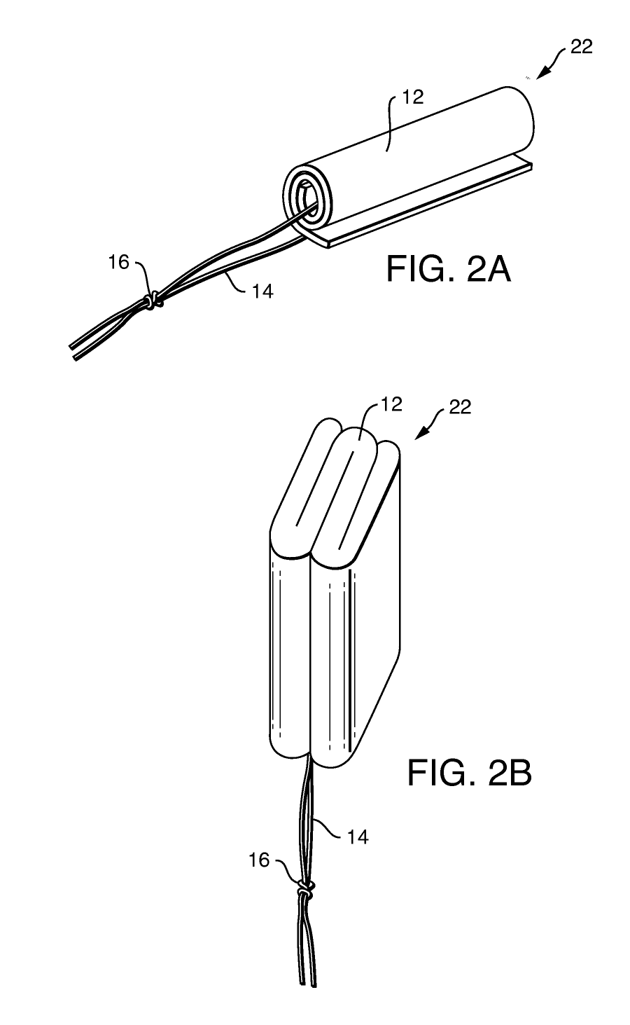

The absorbent structure 10 can be rolled, stacked, folded, or otherwise manipulated into a pledget 22 before compressing the pledget 22 into a tampon 24. FIG. 2A is an illustration of a perspective view of an example of a rolled pledget 22, such as a radially wound pledget 22. FIG. 2B is an illustration of a perspective view of an example of a folded pledget 22. It is to be understood that radially wound and folded configurations are illustrative and additional pledget 22 configurations are possible. For example, suitable menstrual tampons may include “cup” shaped pledgets like those disclosed in U.S. Publication No. 2008/0287902 to Edgett and U.S. Pat. No. 2,330,257 to Bailey; “accordion” or “W-folded” pledgets like those disclosed in U.S. Pat. No. 6,837,882 to Agyapong; “radially wound” pledgets like those disclosed in U.S. Pat. No. 6,310,269 to Friese; “sausage” type or “wad” pledgets like those disclosed in U.S. Pat. No. 2,464,310 to Harwood; “M-folded” tampon pledgets like those disclosed in U.S. Pat. No. 6,039,716 to Jessup; “stacked” tampon pledgets like those disclosed in U.S. 2008/0132868 to Jorgensen; or “bag” type tampon pledgets like those disclosed in U.S. Pat. No. 3,815,601 to Schaefer.

A suitable method for making “radial wound” pledgets is disclosed in U.S. Pat. No. 4,816,100 to Friese. Suitable methods for making “W-folded” pledgets are disclosed in U.S. Pat. No. 6,740,070 to Agyapong; U.S. Pat. No. 7,677,189 to Kondo; and U.S. 2010/0114054 to Mueller. A suitable method for making “cup” pledgets and “stacked” pledgets is disclosed in U.S. 2008/0132868 to Jorgensen.

In various embodiments, the pledget 22 can be compressed into a tampon 24. Additional details regarding an apparatus and method of compression will be provided later herein. The pledget 22 may be compressed any suitable amount. For example, the pledget 22 may be compressed at least about 25%, 50%, or 75% of the initial dimensions. For example, a pledget 22 can be reduced in diameter to approximately ¼ of the original diameter. The transverse configuration of the resultant tampon 24 may be circular, ovular, elliptical, rectangular, hexagonal, or any other suitable shape.

FIG. 3A provides an illustration of an embodiment of a side view of an exemplary tampon 24 having a relatively smooth outer surface. FIG. 3B provides an illustration of an embodiment of a side view of an exemplary tampon 24 having topographical features such as grooves 32 and ribs 34. FIG. 3C provides an illustration of an embodiment of a side view of an exemplary tampon 24 having topographical features such as grooves 32 and indentations 400. FIG. 3D provides an illustration of an embodiment of a side view of an exemplary tampon 24 having topographical features such as grooves 32, indentations 400, and raised rings 402. The tampon 24 can have an insertion end 26 and a withdrawal end 28. The tampon 24 can have a length 36 wherein the length 36 is the measurement of the tampon 24 along the longitudinal axis 30 originating at one end (insertion or withdrawal) of the tampon 24 and ending at the opposite end (insertion or withdrawal) of the tampon 24. In various embodiments, the tampon 24 can have a length 36 from about 30 mm to about 60 mm. The tampon 24 can have a compressed width 38, which unless otherwise stated herein, can correspond to the greatest transverse cross-sectional dimension along the longitudinal axis 30 of the tampon 24. In some embodiments, the tampon 24 can have a compressed width 38 prior to usage from about 2, 5, or 8 mm to about 10, 12, 14, 16, 20 or 30 mm. The tampon 24 can be straight or non-linear in shape, such as curved along the longitudinal axis 30.

In various embodiments, the tampon 24 may be placed into an applicator. In various embodiments, the tampon 24 may also include one or more additional features. For example, the tampon 24 may include a “protection” feature as exemplified by U.S. Pat. No. 6,840,927 to Hasse, U.S. 2004/0019317 to Takagi, U.S. Pat. No. 2,123,750 to Schulz, and the like. In some embodiments, the tampon 24 may include an “anatomical” shape as exemplified by U.S. Pat. No. 5,370,633 to Villalta, an “expansion” feature as exemplified by U.S. Pat. No. 7,387,622 to Pauley, an “acquisition” feature as exemplified by U.S. 2005/0256484 to Chase, an “insertion” feature as exemplified by U.S. Pat. No. 2,112,021 to Harris, a “placement” feature as exemplified by U.S. Pat. No. 3,037,506 to Penska, or a “removal” feature as exemplified by U.S. Pat. No. 6,142,984 to Brown.

### Pessary:

A pessary can be used by a woman in the treatment of urinary incontinence. In various embodiments, the pessary can be adapted to be disposable, worn only for a relatively short period of time and then discarded and replaced with a new pessary (if needed). Alternatively, the pessary can be recycled for use by sterilizing it between uses. The pessary can be simple and easy to use and can, optionally, be inserted in the same user-friendly manner that a tampon is inserted into the vaginal cavity during menstruation, for example either digitally or by using an applicator. In an embodiment, the pessary can be inserted in any orientation since the pessary can naturally migrate into a correct treatment position as a result of the pessary geometry. As with insertion, removal can be accomplished in a similar manner as a tampon, such as by pulling on a withdrawal element.

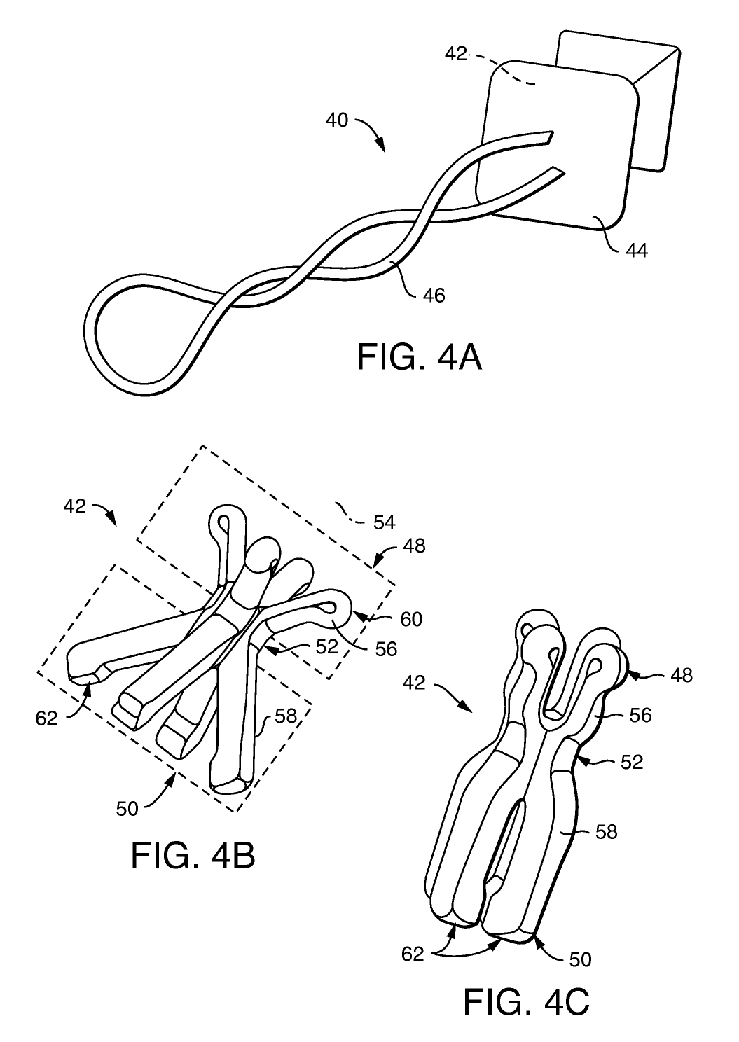

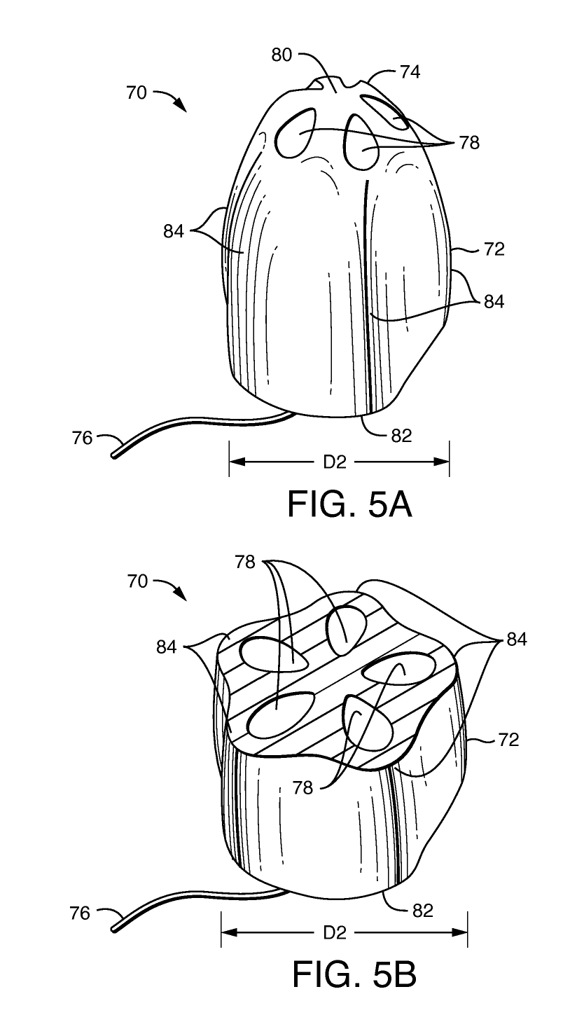

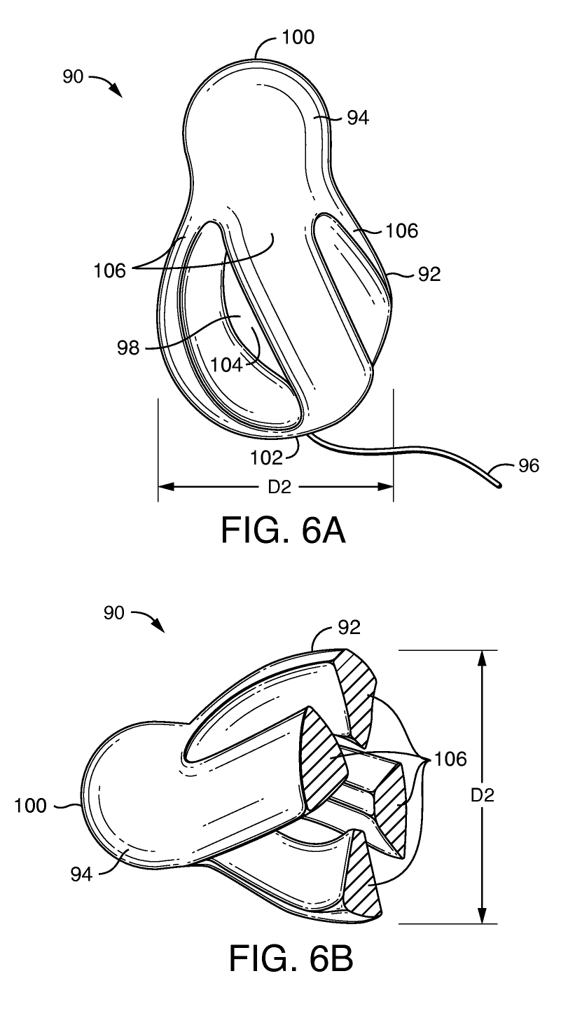

A pessary can be provided in many configurations, each of which can be compressed into a size and dimension more suitable for insertion into the body either digitally by the user's fingers or through the use of an applicator. FIGS. 4A-4C illustrate an exemplary embodiment of a pessary 40 having a core 42, a cover 44, and a withdrawal element 46. FIGS. 5A and 5B illustrate an exemplary embodiment of a pessary 70 having a fold 84. FIGS. 6A and 6B illustrate an exemplary embodiment of a pessary 90 having a strut 106.

An example of an embodiment of a pessary 40 having a core 42, a cover 44, and a withdrawal element 46 can be seen in FIG. 4A. Referring to FIG. 4B, a perspective view of an exemplary embodiment of a core 42 for the pessary 40 is illustrated. For ease of description, the core 42 can be arranged around a longitudinal axis 54 and divided into three basic elements. A top section 48, inside the dashed box, can be provided which can serve as the “anchoring” element for stabilizing the pessary 40 within the vagina. A bottom section 50, inside the dashed box, can be provided which can serve as the “supporting” element for generating support. In various embodiments, support can be generated at a sub-urethral location, for example mid-urethra. In various embodiments, the roles of anchoring 48 and supporting 50 elements can be switched or shared. In an embodiment, the anchoring 48 and supporting 50 elements of the core 42 can function as an internal support structure for a cover 44. In an embodiment, an intermediate section can be provided which can act as a “node” 52 and which can connect anchoring 48 and supporting 50 elements. The node 52 of core 42 can have a length which can be a small portion of the overall length of the core 42. In various embodiments, the length of the node 52 can be less than about 15, 20 or 30% of the entire length of the core 42.

In an exemplary embodiment, the anchoring element 48 and the supporting element 50 can each have four arms, 56 and 58, respectively. In such an exemplary embodiment, two arms, 56 and 58, of each of the anchoring 48 and supporting 50 elements, respectively, can generally exert pressure towards the anterior vaginal wall and two arms, 56 and 58, of each of the anchoring 48 and supporting 50 elements, respectively, can generally exert pressure towards the posterior vaginal wall adjacent the bowels. The distal part of the urethra extends into the vagina forming a recess between the urethral bulge and the vaginal wall. The arms, 56 and/or 58, which exert pressure anteriorly can fit within these natural recesses on either side of the urethra.

In various embodiments, the anchoring element 48 and the supporting element 50 can each have more or less arms, 56 and 58, respectively. For example, the anchoring element 48 could have more anchoring arms 56 if there is concern about unwanted movement of the pessary 40.

Referring to FIG. 4B, the anchoring arms 56 can have tips 60 and the supporting arms 58 can have tips 62. In various embodiments, the tips 60 of the anchoring arms 56 can be rounded or spherical in nature, to provide smooth surfaces (i.e., no corners or points) for the tenting of the vaginal wall. In various embodiments, the tips 62 of the supporting arms 58 and/or corners of core 42 can be blunted by a beveled edge both along the anchoring arms 56 and supporting arms 58 and at the tips 62, such as shown in FIG. 4B. In an embodiment, the beveled edge of the supporting arms 58 can reduce the overall circumference of the core 42, relative to a completely spherical cross section, when it is in a compressed mode for packaging within an applicator. An example of an inwardly compressed core 42 can be seen in FIG. 4C.

In various embodiments, the core 42 can be made in a plurality of sizes and/or to exhibit specific performance characteristics, such as radial expansion of the supporting arms 58. In various embodiments, the diameter of a radially expanded anchoring element 48 can range from about 30 to about 33 mm. In various embodiments, the diameter of a radially expanded supporting element 50 can range from about 34 mm to about 52 mm. In various embodiments, the core 42 can also be made of different materials and/or materials exhibiting different performance characteristics, such as, for example, hardness. In various embodiments, the core 42 can be constructed of a material or materials which can exhibit a Shore A hardness of 30-80. In various embodiments, core 42 can be manufactured in multiple Shore A hardnesses, including, but not limited to, 40, 50 and 70.

In various embodiments, the core 42 can be constructed from a single piece (Monoblock). In various embodiments, the core 42 can have an anchoring element 48 and a supporting element 50 which can be provided as separate pieces (bi-polar) which can be attached to form the core 42. In various embodiments, each element, supporting 50 or anchoring 48, can be constructed of two or more pieces. In various embodiments, core 42 can be constructed of liquid silicone (LSR) by injection molding. It is possible to use other materials, for example TPE, non-liquid silicone, and others for a core 42 of the same size. In an embodiment, materials exhibiting various degrees of Shore A hardness can be used to produce softer or more rigid cores 42.

Referring to FIG. 4A, a perspective view of a core 42 enclosed within a cover 44 provided with a withdrawal element 46 is illustrated, in accordance with an exemplary embodiment of the pessary 40. Cover 44 can be optionally any of the covers described in PCT/IL2004/000433; PCT/IL2005/000304; PCT/IL2005/000303; PCT/IL2006/000346; PCT/IL2007/000893; PCT/IL2008/001292. In various embodiments, the cover 44 and the withdrawal element 46 can be constructed of the same unitary piece of material and/or at the same time and/or in the same process. In various embodiments, the cover 44 and the withdrawal element 46 can be constructed of separate pieces of material.

In various embodiments, the withdrawal element 46 can be constructed of a cotton material but can be constructed of other materials such as are known to one of ordinary skill in the art. In various embodiments, the withdrawal element 46 of the pessary 40 can be from about 14 cm to about 16 cm in length, although the length can be varied in different pessary 40 configurations. In an embodiment, the withdrawal element 46 can be secured to the cover 44 in a position whereby a pulling force towards the vaginal introitus can be substantially evenly distributed over the cover 44 as it collapses the supporting arms 58 of the core 42 within the vagina. In an embodiment, this position can be in the center of the cover 44 in the supporting element 50 region, such as illustrated in FIG. 4A.

Referring to FIGS. 5A and 5B, an illustrative example of another embodiment of a pessary 70 is shown. The pessary 70 includes a supporting element 72, an anchoring element 74, a withdrawal element 76, and at least one fluid passageway 78 extending though the pessary 70. The pessary 70 has a distal end 80 and a proximal end 82. The distal end 80 refers to that portion of the pessary 70 that is first inserted into the vagina. The pessary 70, not including the withdrawal element 76 may have a length of from about 10, 30 or 50 mm to about 70, 90 or 120 mm.

The pessary 70 can have a different configuration depending on whether the pessary 70 is being inserted, is in-use, or being removed. When the pessary 70 is in-use, the supporting element 72 of the pessary 70 can have a generally conical shape (such as illustrated in FIG. 5A). The supporting element 72 can expand from a compressed configuration and into the conical shape as the pessary 70 is inserted into the vaginal cavity. While the supporting element 72 is described as being conically shaped, it may also be shaped in the form of a pear, a tear drop, an obconical, or similar shape. Accordingly, the term “conical shape” is meant to include a shape as depicted in FIG. 5A, as well as a pear shape, a tear drop shape, an obconical, or similar shape. Typically, the proximal end 82 of the pessary 70 will have a largest outer circumference with an in-use diameter, D2, which is larger than any other point on the supporting element 72. In an embodiment, the in-use diameter, D2, can range from about 20 or 40 mm to about 50 or 60 mm.

The pessary 70 may have a plurality of folds 84 extending from the distal end 80 to the proximal end 82. In an embodiment, the number of folds 84 extending from the distal end 80 to the proximal end 82 can be from 2 or 4 to 6. FIGS. 5A and 5B illustrate a pessary 70 having 5 folds 84. Prior to insertion, the pessary 70 can be in a compressed configuration and the folds 84 can be compressed or folded inward. When the plurality of folds 84 are compressed and folded inward, the largest outer circumference of the pessary 70 may have an insertion diameter which allows for easier insertion into the vagina. The insertion diameter can be smaller than the in-use diameter, D2. In an embodiment, the insertion diameter can range from 10 or 15 mm to about 20 or 25 mm.

The pessary 70 can have a fluid passageway 78 which can serve at least one of two functions. First, the fluid passageway 78 can provide the space necessary in the pessary 70 to allow for the folds 84 to compress inward to provide the pessary 70 with its insertion diameter. Secondly, the fluid passageway 78 can facilitate the natural movement of vaginal fluids entering the pessary 70. In an embodiment, there can be a fluid passageway 78 for each fold 84.

As discussed above, an anchoring element 74 can be located at the distal end 80 of the pessary 70. The anchoring element 74 can prevent the pessary 70 from unintentionally moving, thereby stabilizing the pessary 70 within the vaginal cavity. In an embodiment, the anchoring element 74 may have a diameter ranging from about 10 or 15 mm to about 20 or 25 mm.

Referring to FIGS. 6A and 6B, an illustrative example of another embodiment of a pessary 90 is shown. The pessary 90 includes a supporting element 92, an anchoring element 94, a withdrawal element 96 and at least one fluid passageway 98 extending through the pessary 90. The pessary 90 has a distal end 100, a proximal end 102, and a hollow interior section 104. The distal end 100 refers to that portion of the pessary 90 that is first inserted into the vagina. The pessary 90, not including the withdrawal element 96, may have a length of from about 10, 30 or 50 mm to about 70, 90 or 120 mm.

The pessary 90 can have a different configuration depending on whether the pessary 90 is being inserted, is in-use, or being removed. When the pessary 90 is in use, the pessary 90 can have a generally convex shape (such as illustrated in FIG. 6A). The supporting element 92 can expand from a compressed configuration and into the convex shape as the pessary 90 is inserted into the vaginal cavity. The convex shape of the supporting element 92 can provide the necessary support to the vaginal walls by contacting with an anterior vaginal wall and a posterior vaginal wall. While the supporting element 92 is described as being a convex shape, it may also be shaped in the form of a pear, a tear drop, an oval or similar shape. Accordingly, the term “convex shape” is meant to include a shape as depicted in FIG. 6A, as well as a pear shape, a tear drop shape, an oval, or similar shape. In an embodiment, the supporting element 92 can have an in-use diameter, D2, ranging from about 20 or 40 mm to about 50 or 60 mm.

The supporting element 92 can have a plurality of struts 106 extending from the distal end 100 to the proximal end 102. In an embodiment, the number of struts 106 extending from the distal end 100 to the proximal end 102 can be from 2, 3 or 4 to 5 or 6. FIGS. 6A and 6B illustrate a pessary 90 having 4 struts 106. Prior to insertion, the pessary 90 can be in a compressed configuration and the struts 106 can be twisted together and compressed. As a result of twisting and compressing the struts 106, the pessary 90 can lengthen. When the struts 106 are twisted together, a largest circumference of the supporting element 92 can have an insertion diameter that allows for easier insertion into the vagina. The insertion diameter also allows for insertion and storage within an applicator. The insertion diameter can be smaller than the in-use diameter, D2, and can range from about 10 or 15 mm to about 20 or 25 mm.

The pessary 90 can have a hollow interior section 104 which can serve at least one of two functions. First, the hollow interior section 104 can provide the space necessary in the pessary 90 to allow for the struts 106 to twist together, nest and compress to provide a the pessary 90 with its insertion diameter. Secondly, the hollow interior section 104 can provide a fluid passageway 98 to facilitate the transport of any fluids entering the pessary 90.

As discussed above, an anchoring element 94 can be located at the distal end 100 of the pessary 90. The anchoring element 94 can prevent the pessary 90 from unintentionally moving, thereby stabilizing the pessary 90 within the vaginal cavity. In an exemplary embodiment, the anchoring element 94 does not apply significant pressure to the wearer's vagina and/or urethra, thereby enhancing comfort. In an embodiment, the anchoring element may have a diameter ranging from about 10 or 15 mm to about 20 or 25 mm.

In addition, the pessaries, 70 and 90, can each have a withdrawal element, 76 and 96, respectively, attached to the pessary, 70 and 90, respectively. The withdrawal element, 76 and 96, may be a separate piece or may be integrally formed with the pessary, 70 or 90, respectively. Pulling on the withdrawal element, 76 or 96, may cause the supporting element, 72 or 92, to inwardly collapse upon itself to reduce the largest circumference of the cross-sectional area of the supporting element, 72 or 92, of the pessary, 70 or 90, respectively, for easier removal.

The pessary, 70 or 90, can comprise a compliable resilient material. As used herein, the term “resilient material” and variants thereof relate to materials that can be shaped into an initial shape, which initial shape can be subsequently formed into a stable second shape with mechanical deformation such as bending, compressing or twisting the material. The resilient material then substantially reverts to its initial shape when the mechanical deformation ends. The pessary, 70 or 90, can be formed initially in the in-use configuration as described above. The pessary, 70 or 90, can then be compressed for insertion or storage within an applicator. After the pessary, 70 or 90, is inserted, the pessary, 70 or 90, can transition from the compressed configuration to the in-use configuration due to the ability of the resilient material to relax or spring back to its original shape.

The pessary, 70 or 90, may also be covered with a suitable biocompatible cover material such as is known to one of ordinary skill in the art. The pessary, 70 or 90, may be enclosed in a cover that may reduce friction during deployment, help control the pessary, 70 or 90, during insertion and removal, help the pessary, 70 or 90, to stay in place, and/or create more contact area for applying pressure to the vaginal walls.

### Apparatus:

The present disclosure is generally directed towards an apparatus which can be used in the compression step of a manufacturing process of a tampon (such as, for example, tampon 24 illustrated in FIGS. 3A-3D) or pessary (such as, for example, pessary 40, 70 or 90 illustrated in FIGS. 4A-4C, 5A, 5B, 6A, and 6B). The apparatus can have a plurality of press unit support structures which can each be capable of carrying at least one press unit. Each individual press unit can compress a material, such as, for example, a pledget or an uncompressed pessary. As the apparatus can have a plurality of individual press units, the apparatus can compress more than one material at a time.

Each press unit support structure can be capable of rotating about an axis. In various embodiments, the rotation of each press unit support structure about an axis can occur continuously. In various embodiments, the rotation of each press unit support structure about an axis can occur intermittently. As each individual press unit support structure rotates about the axis, each press unit carried by each press unit support structure also rotates about the axis. The rotation of a press unit support structure can occur independently of any other press unit support structure. Each press unit support structure can experience a variation in speed during a single revolution about the axis. Therefore, at any moment in time, a press unit support structure may be rotating at a speed which can vary from the speed of another press unit support structure in a single revolution of the press unit support structures about the axis. The spatial relationship, therefore, between an individual press unit carried on a press unit support structure and another press unit carried on a second press unit support structure can vary.

In various embodiments, an apparatus can carry a plurality of individual press units. In various embodiments, the apparatus can carry at least 2, 3, 4, 5, 6, 7, 8, 9 or 10 press units. In various embodiments, the apparatus can carry from 2, 3, 4 or 5 press units to 6, 7, 8, 9, or 10 press units. Each press unit can be releasably secured to its respective press unit support structure. As each press unit can be releasably secured to a press unit support structure, should a press unit malfunction, the operation of the apparatus can be stopped, the press unit can be removed from the press unit support structure by disengaging releasable mounts (such as bolts or pins), and the malfunctioning press unit can be replaced with a working press unit.

During a single revolution of a press unit support structure about an axis, each individual press unit positioned on a press unit support structure can undergo a complete compression cycle in order to compress a material located within the chamber of the press unit. The compression cycle can begin with the loading of an uncompressed material into an individual press unit which can be in a full open configuration. The full open configuration of the press unit can provide a chamber into which the material can be loaded. Following the loading of a material into the chamber of the press unit, the press unit can begin to transition from the full open configuration, through a partially closed configuration, and to a full closed configuration. Compression of the material within the chamber can begin during the transition from the full open configuration of the press unit to the full closed configuration of the press unit as the volume of the chamber is decreasing during this transition. Once the press unit has reached the full closed configuration, the press unit can dwell in the full closed configuration for as long of time during the single revolution of the press unit support structure about the axis as deemed suitable. The length of dwell can impact the ability of the material under compression to maintain a compressed configuration upon removal of the compression pressure. When the material in the chamber has been compressed to the desired level of compression, the press unit can begin to transition from the full closed configuration, through a partially open configuration, and to a full open configuration. As the press unit transitions from the full closed configuration to a full open configuration, the volume of the chamber can increase. As the material in the chamber was recently undergoing compression, the material may begin to rebound from the compression and expand as the compression pressure is decreased. To minimize the expansion of the material to its original starting dimensions, in various embodiments, the material may be unloaded from the chamber while the press unit is in a partially open configuration. In various embodiments wherein the compressed material is stable in the compressed configuration, the unloading of the material from the chamber can occur when the press unit has reached the full open configuration. Following the unloading of the compressed material from the chamber of the press unit, the press unit can repeat the compression cycle in a new revolution of the press unit support structure about the axis. During a compression cycle, and in a single revolution of the press unit support structure about an axis, a press unit can transition from a full open configuration, through a partially closed configuration to a full closed configuration and, from the full closed configuration through a partially open configuration to a full open configuration.

The length of time for a press unit to remain in each configuration (e.g., full open, partially closed, full closed, partially open) can be any length of time during the single revolution about the axis as deemed suitable to compress the material to the desired size dimensions and desired compressed stability. The dwell time of a material in a press unit in a full closed configuration during the compression cycle can be, therefore, any length of time as deemed suitable to compress the material to the desired size dimensions and desired compressed stability. In various embodiments, in a single revolution of each press unit support structure about an axis, a material to be compressed can be loaded into a press unit in a full open configuration, the material can be compressed, and the compressed material can be unloaded from the press unit after the press unit has completed about 90, 120, 150, 180, 210, 240, 270, 300, 330 or 360 degrees of rotation ±10° about the axis around which the press unit support structure rotates. Compression of a material within a press unit can begin at any point after the loading of the material into the press unit and can continue until the press unit has rotated at least about 90, 120, 150, 180, 210, 240, 270, 300, 330 or 360 degrees of rotation ±10° about the axis. For example, an apparatus can have three press unit support structures and each press unit support structure can carry a press unit. In such an example, a material can be loaded into a press unit of a press unit support structure, can undergo compression, and can be unloaded from such press unit at about the 120, 240 or 360 degree position ±10°. It is to be understood that more or fewer press unit support structures can alter the degree positions at which a material can be unloaded from a press unit.

In various embodiments, an apparatus can carry a press unit which can compress a material in an axial direction. In various embodiments, an apparatus can carry a press unit which can compress a material in a non-linear direction, such as, for example, compression in an arcuate motion in a predominantly radial direction. In various embodiments, an apparatus can carry a press unit which can have a compression surface area which can decrease with the movement of compression. In various embodiments, an apparatus can carry a press unit which can have the capability to compress a material utilizing two types of compression (i.e., axial direction compression, non-linear direction compression, and/or with a decreasing compression surface area). As a non-limiting example, in an embodiment, an apparatus can carry a press unit which can compress a material in an axial direction and can also compress the same material in a non-linear direction. In such an embodiment, the axial direction compression can occur prior to or after the non-linear direction compression.

In various embodiments, an apparatus can carry at least two axial direction press units. In various embodiments, an apparatus can carry at least two non-linear direction press units. In various embodiments, an apparatus can carry at least two press units which can each have a compression surface area which can decrease with the movement of compression. In various embodiments, an apparatus can carry at least two press units which can each have a capability to provide two types of compression to a material. In various embodiments, an apparatus can carry at least two press units which can each provide a type of compression different than the other press unit. In an embodiment, an apparatus can carry at least two press units wherein one press unit can provide compression in an axial direction and another press unit can provide compression in a non-linear direction or can have a compression surface area which can decrease with the movement of compression. In an embodiment, an apparatus can carry at least two press units wherein one press unit can provide compression in a non-linear direction and another press unit can provide compression in an axial direction or can have a compression surface area which can decrease with the movement of compression. In an embodiment, an apparatus can carry at least two press units wherein one press unit can have a compression surface area which decreases with the movement of compression and another press unit can provide compression in an axial direction or in a non-linear direction.

In various embodiments, the dwell time for a press unit can be varied. For example, in various embodiments it may be desirable to compress a material for a longer period of time. Increasing the dwell time that a material is compressed can increase the stability of the compressed material. For example, in various embodiments, the apparatus can have multiple press unit support structures wherein each press unit support structure can carry a press unit. Each press unit support structure can rotate about an axis independently of any other press unit support structure. The press unit support structures can be configured to rotate about the axis through periods of low dwell speed, acceleration, high dwell speed, and deceleration. The dwell time of compression, therefore, can be varied.

In various embodiments, the compression step may occur without any application of heat to the material, such as a pledget or pessary. In other words, the material can be compressed without external heat being applied to the apparatus or the material. In various embodiments, the compression step can include the application of heat to the material. In other words, the material can be compressed with external heat being applied to the apparatus or the material. In various embodiments, the compression step may incorporate or may be followed by one or more additional stabilization steps. This secondary stabilization can serve to maintain the compressed shape of the tampon or pessary.

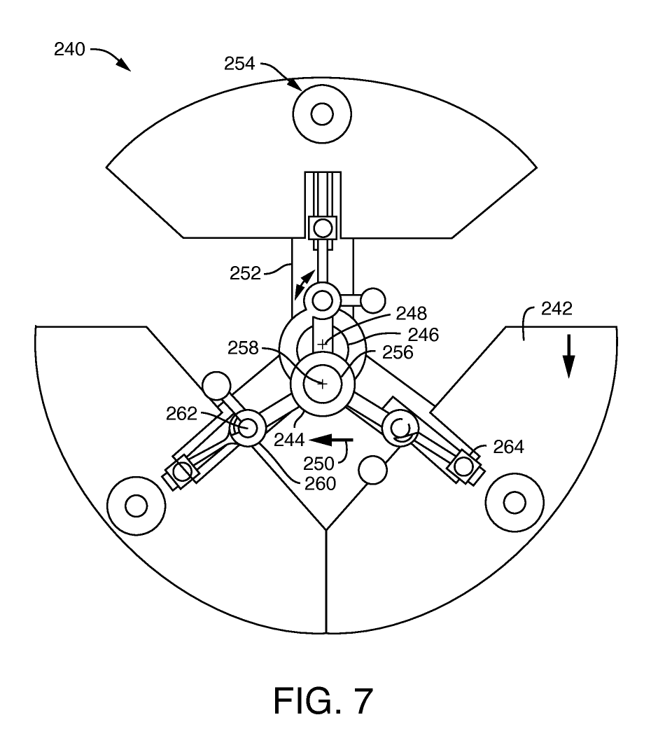

Referring to FIG. 7, a schematic example of an embodiment of an apparatus 240 is illustrated. The apparatus 240 can have a plurality of press unit support structures 242 and each press unit support structure 242 can carry a press unit 254. The illustrated example of apparatus 240 provides three press unit support structures 242. It should be readily understood that the apparatus 240 can include any number of press unit support structures 242. Each press unit support structure 242 can be configured to be rotated by a drive ring 244 and can be coaxially supported and rotatably connected to a common idler shaft 246 on a first axis 248. The press unit support structures 242 can be configured to rotate about the first axis 248 in the direction indicated by arrow 250. Each press unit support structure 242 can include a support member 252 which can be rotatably connected to the idler shaft 246 such that each press unit support structure 242 can be rotated independently. The radial inner end of the support member 252 of each press unit support structure 242 can be rotatably connected to the idler shaft 246 by any technique known to one of those skilled in the art, including, for example, using conventional bearings.

The apparatus 240 can comprise a drive ring 244 which can be configured to rotate each press unit support structure 242 at a variable speed. The inner radial end of the drive ring 244 can be rotatably connected to a fixed shaft 256 on a second axis 258. The drive ring 244 can be configured to be rotated at a constant or variable speed about the second axis 258 by a driving means in the direction indicated by the arrow 250. The driving means can include a motor operatively connected through suitable gearing and drive belts to the drive ring 244. Therefore, in use, the motor can rotate the drive ring 244, which, in turn, can rotate the press unit support structures 242 at the desired speed. To provide a variable speed of each press unit support structure 242, the second axis 258 of the drive ring 244 can be offset from the first axis 248 of the press unit support structures 242. The offset distance between the first axis 248 and the second axis 258 can be any distance which can provide the desired variations in the speed of the press unit support structures 242.

The apparatus 240 can have at least one coupler arm 260 which can be pivotally connected to the drive ring 244 about a pivot point 262. The apparatus 240 can have one coupler arm 260 for each press unit support structure 242. The coupler arm 260 can independently connect the drive ring 244 to each respective press unit support structure 242. Each coupler arm 260 can have a cam end and a crank end 264 which extend radially outward from the pivot point 262. The cam end and the crank end 264 are designed to remain at a fixed angle relative to each other. For example, a first line extending through the pivot point 262 and the cam end and a second line extending through the pivot point 262 and the crank end 264 may define an angle from about 30 degrees to about 180 degrees to provide variable speed. The cam end can follow a predetermined curvilinear path and the crank end 264 can be slidably connected to the respective press unit support structure 242. As the drive ring 244 is rotated, the cam end of each coupler arm 260 is guided along the curvilinear path and the crank end 264 of each coupler arm 260 slidably engages the respective press unit support structure 242 thereby pivoting the coupler arm 260 about the pivot point 262. The pivoting of the coupler arm 260 and the offset crank motion of the drive ring 244 vary the effective drive radius of each press unit support structure 242 and rotate each press unit support structure 242 at variable speed. The press unit support structures 242 can, therefore, be configured to rotate through periods of low dwell speed, acceleration, high dwell speed, and deceleration. A press unit 254 can also, therefore, experience variations in their spatial relationship to other press units 254 carried by the apparatus 240.

In various embodiments, each press unit carried by a press unit support structure can be in a different configuration of the compression cycle than other press units carried by other press unit support structures. In such embodiments, each press unit can be experiencing a different configuration of the compression cycle at any moment in time during the revolution of the various press unit support structures about the axis. For example, in a revolution of a press unit support structure about an axis, at an initial moment in time, a material can be loaded into a first press unit. The press unit support structure can continue to rotate about the axis and the first press unit can transition from a full open configuration, through a partially closed configuration and to a full closed configuration to compress the material loaded within the first press unit. While the first press unit is undergoing the transition from the full open configuration to the full closed configuration, a second material can be loaded into a second press unit carried by a second press unit support structure for compression. It should be understood that the second material can be loaded into the second press unit while the first press unit is in any of the configurations of the compression cycle. As the press units can be in different configurations during revolution about the axis, it can be possible, in various embodiments, to load a material for compression into one press unit at substantially the same time as a compressed material is being unloaded from another press unit. In various embodiments, during a revolution of the various press unit support structures about an axis, each press unit can be operated an actuated independently of any other press unit carried by the apparatus. In other words, each press unit can be out of phase with each other press unit. When the press units are out of phase with each other, they can each be experiencing a different configuration of the compression cycle at any moment in time.

In various embodiments, during a revolution of the multiple press unit support structures about an axis, each press unit can be operated and actuated substantially synchronously with each other press unit carried by the apparatus. In other words, each press unit can be in phase with each other press unit. When the press units are in phase with each other, they can each undergo the configurations of the compression cycle substantially in synchronicity with each other press unit. For example, in a revolution of the multiple press unit support structures about the axis, each press unit can have a material loaded into the press unit at substantially the same time when the press units are in the full open configuration of the compression cycle. The press unit support structures can continue to rotate about the axis, and each press unit can transition from the full open configuration to the full closed configuration at substantially the same time. The press unit support structures can continue to rotate about the axis and following compression of the material in each press unit, the press units can transition from the full closed configuration to the full open configuration. As described above, the compressed material can be unloaded from the press units during the transition from the full closed configuration to the full open configuration, i.e., in the partially open configuration, or when the press units have reached the full open configuration. In various embodiments, at a moment in time during the revolution of the press unit support structures about the axis, at least two press units can be in a full open configuration. In various embodiments, at a moment in time during the revolution of the press unit support structures about the axis, at least two press units can be in a partially closed configuration. In various embodiments, at a moment in time during the revolution of the press unit support structures about an axis, at least two press units can be in a full closed configuration. In various embodiments, at a moment in time during the revolution of the press unit support structures about an axis, at least two press units can be in a partially open configuration.

In various embodiments, in a moment of time during a revolution of at least two press unit support structures about an axis, a first press unit carried by one of the press unit support structures of the apparatus can be in one of a full open configuration, a partially closed configuration, a full closed configuration, or a partially open configuration and a second press unit carried by a second of the press unit support structures of the apparatus can be in one of a full open configuration, a partially closed configuration, a full closed configuration, or a partially open configuration. In such an embodiment, the configuration of the first press unit of the apparatus can be the same as or can be different than the configuration of the second press unit of the apparatus. In various embodiments, an additional press unit(s) can be carried by the apparatus. In such various embodiments, in a moment of time during a revolution about an axis of another press unit support structure carrying the additional press unit, the additional press unit(s) of the apparatus can be in a configuration (full open, partially closed, full closed, or partially open) which can be the same as or different than at least one other press unit carried by the apparatus.

As described above, an apparatus 240 (or such similar apparatus) can carry a plurality of press units 254 to compress a material, such as, for example, a pledget or an uncompressed pessary. As described above, a press unit 254 can provide compression in the axial direction, in a non-linear direction, can have a compression surface area which decreases during the movement of compression, or can provide a combination of these types of compression. The press unit 254 can, therefore, be in the form of an axial direction press unit, a non-linear direction press unit, a decreasing compression surface area press unit, or a combination thereof. For clarity of description, the disclosure herein may refer only to the compression of a pledget. It is to be understood, however, that the compression described can be applied to a pessary.

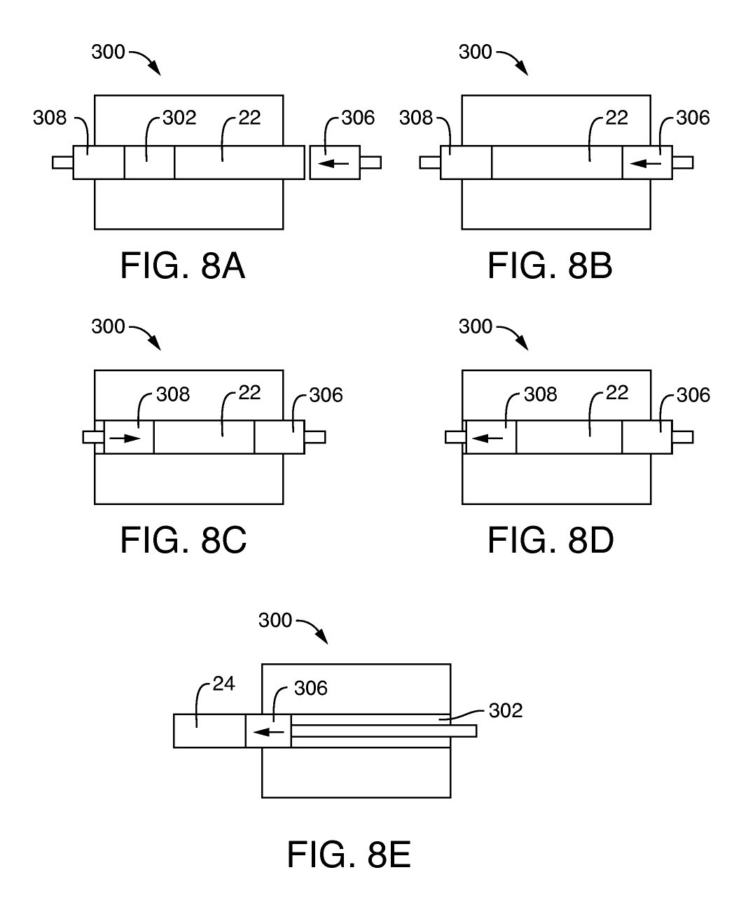

Compression in the axial direction can compress a material, such as a pledget or pessary, in the longitudinal direction, lateral direction, or both the longitudinal and lateral directions. Referring to FIGS. 8A-8E, a schematic illustration of an exemplary embodiment of compression of a material in the longitudinal direction by use of an axial direction press unit 300 is presented. A pledget 22 can be introduced into a compression chamber 302 of the axial direction press unit 300 (such as shown in FIG. 8A). The pledget 22 can be urged into the chamber 302 by a reciprocating push rod 306. The pledget 22 can be pushed into the chamber until it reaches the end of the chamber 302, which can correspond to the face of a reciprocating piston 308 (such as shown in FIG. 8B). After the pledget 22 has been pushed into the chamber 302, the chamber 302 can be closed. Closing of the chamber 302 can be affected by having the push rod 306 and piston 308 remain at least partially within the chamber 302 thereby closing any openings to the chamber 302. It will be understood that alternate means can close the chamber 302, such as, for example, separate closing means can be provided. After the pledget 22 has been fully inserted into the chamber 302, the pledget 22 can be compressed in the longitudinal direction by utilizing the piston 308 to apply a force against the end of the pledget 22 (such as shown in FIG. 8C). Once the pledget 22 has been compressed to the desired longitudinal length, the compression force can be released by withdrawing the piston 308 from the chamber 302 (such as shown in FIG. 8D). A tampon 24 can then be dispelled from the chamber 302. In an embodiment (such as shown in FIG. 8E), the push rod 306 can push the tampon 24 from the chamber 302.

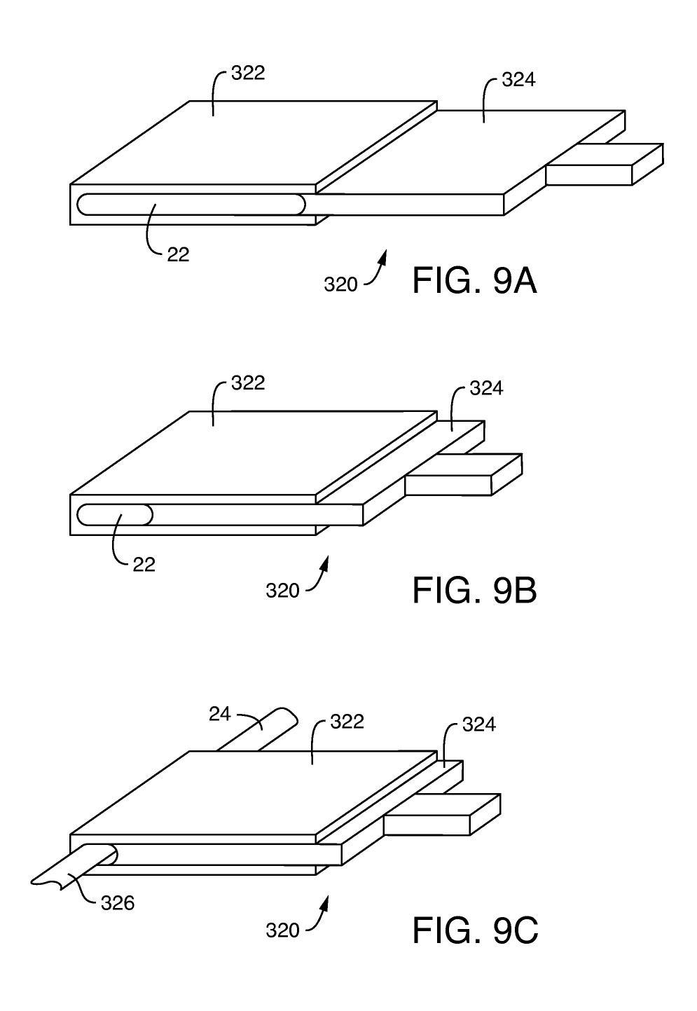

Referring to FIGS. 9A-9C, a schematic illustration of an exemplary embodiment of compression of a material in the lateral direction by use of an axial direction press unit 320 is illustrated. A pledget 22 can be introduced into a compression chamber 322 of the axial direction press unit 320. The pledget 22 can be urged into the chamber 322 by a reciprocating push rod 324. The pledget 22 can be pushed into the chamber 322 until it reaches the end of the chamber 322 (such as shown in FIG. 9A). After the pledget 22 has been fully inserted into the chamber 322, the pledget 22 can be compressed in the lateral direction by using the push rod 324 to apply a force against the pledget 22 (as shown in FIG. 9B). Once the desired width has been achieved, a tampon 24 can be dispelled from the chamber 322 by using a piston 326 to push the tampon 24 from the chamber 322 (such as shown in FIG. 9C). While only one push rod 324 is illustrated in FIGS. 9A-9C, it is to be understood that an axial direction press unit compressing a material in a lateral direction can utilize more than one push rod. For example, multiple push rods can be positioned radially around a material, such as a pledget or uncompressed pessary, which can apply a lateral direction compression against the material during compression. An exemplary apparatus having multiple push rods which are positioned radially around a material and which can apply lateral direction compression against the material during compression is disclosed in U.S. Pat. No. 2,798,260 to Niepmann, the disclosure of which is hereby incorporated by reference in its entirety.

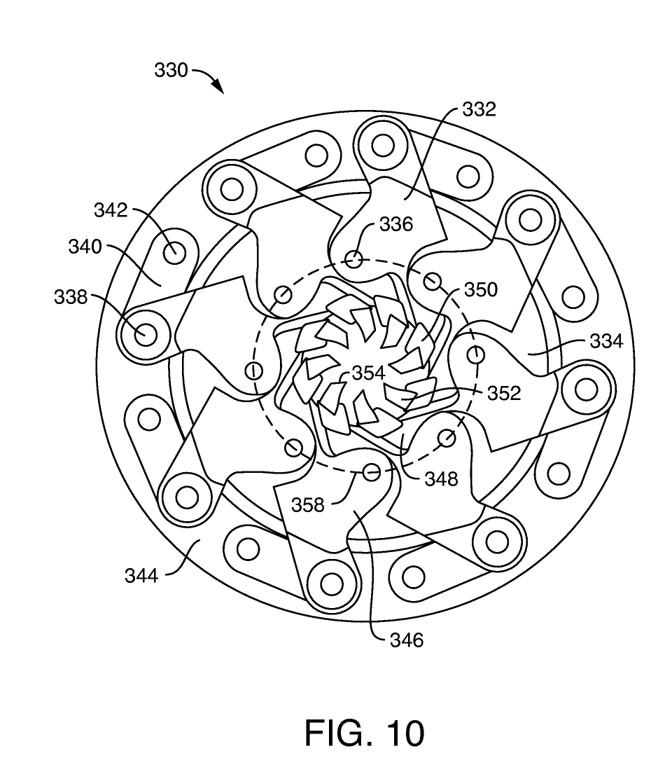

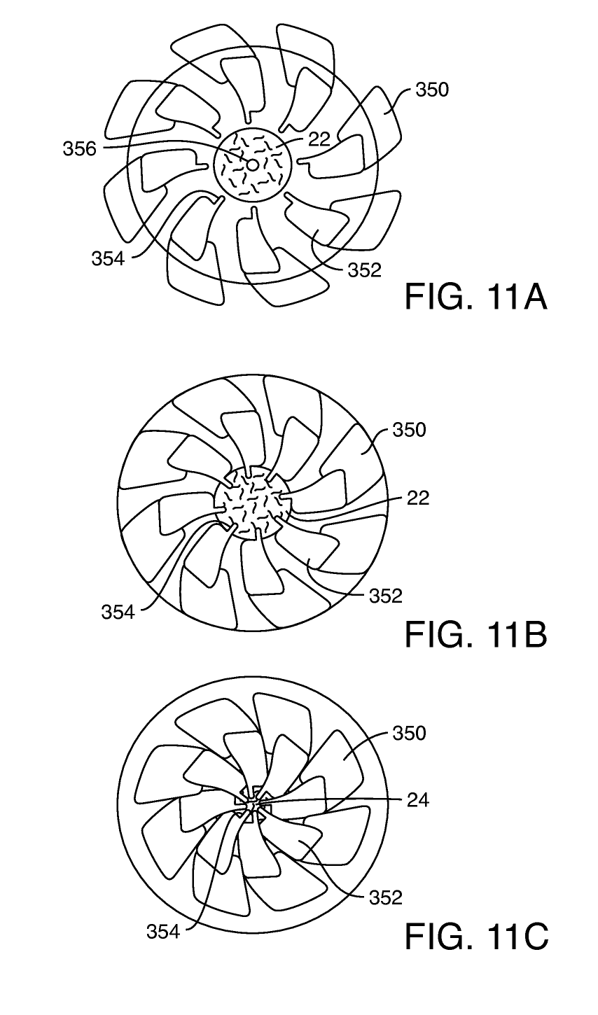

Referring to FIGS. 10 and 11A-11C, a schematic illustration of an exemplary embodiment of a non-linear direction press unit 330 is illustrated. The non-linear direction press unit 330 can have, for example, eight levers 332 each supported at an adjusting ring 334 and pivotable within certain limits about a bearing pin 336. At its radially outer end each lever 332 can be pivotably linked by a coupling pin 338 to a coupling lever 340, the other end of which can be pivotably supported by means of a pin 342 at a stationary ring bearing 344. The pins 342 as well as the bearing pins 336 can each be positioned on a circle, whereby the spacing of these bolts toward one another can be a result of the sectioning specified by the number of levers 332 on the respective circle.

The levers 332, which can be designed as angle levers and which can be provided with a projecting portion 346 between their support location by the bearing pin 336 on the adjusting ring 334 and their articulation by a coupling pin 338 on the coupling lever 340, furthermore comprise a lever arm 348 that can be positioned radially inwardly and supports at its end portion that is positioned radially inwardly a tool carrier 350 to which a pressing tool 352 can be attached. Each pressing tool 352 can be provided with a pressing edge 354.

By rotating the adjusting ring 334 that can be concentrically arranged with respect to the stationary ring bearer 344, a swiveling of the lever 332 can be caused. On rotating the adjusting ring 334 counterclockwise, these levers 332 can be moved radially inwardly with their pressing tools 352. Thus, the levers 332 swivel about the bearing pins 336 which can be arranged at the adjusting ring 334 whereby the coupling pins 338 that are connected with the stationary ring bearing 344 via the coupling levers 340 produce the swiveling movement which results in a radially inwardly directed movement of the pressing tools 352. Thus, a “closing” of the pressing tools 352 is performed. When the adjusting ring 334 is rotated clockwise, an “opening” of the pressing tools 352 is performed.

FIG. 11A illustrates that in the open starting position the pressing edges 354 are not directed towards the center of the non-linear direction press unit 330 but tangentially toward a circular cylinder 356 that surrounds the longitudinal center axis. Thus, it is achieved that the pressing forces which are applied by the pressing tools 352 are not centrally but tangentially directed toward a circle that surrounds the longitudinal center axis of the tampon 24 to be manufactured. This eccentric orientation of the pressing tools 352 toward the central point of the non-linear direction press unit 330 can be adjusted to any desired position by respectively positioning the bearing pin 336 and by providing a corresponding design of the levers 332 as well as of the coupling levers 340.

In the open starting position of the non-linear direction press unit 330, a pledget 22 can be inserted into the opening between the pressing tools 352 (such as illustrated in FIG. 11A). By rotating the adjusting ring 334 counterclockwise relative to the stationary ring bearing 344, the pressing tools 352 are first brought into a partially closed position (such as illustrated in FIG. 11B). With this swiveling movement, the levers 332 are moved with the adjusting ring 334 and are swiveled about the bearing pins 336 of the rotating adjusting ring 334 by the coupling levers 340 that are articulated at the stationary ring bearing 344 such that the pressing tools 352 perform a movement combined of a tangential and a radial component. During this movement, the deformation forces which are applied by the pressing tools 352 and their pressing edges 354 lead to a volume reduction of the pledget 22 that is uniform about the periphery and transforms the pledget 22 into a tampon 24 having a core and ribs and grooves which surround the core (such as illustrated in FIG. 11C). Referring to FIG. 3B, a tampon 24 is illustrated having ribs 34 and grooves 32.

In various embodiments, it may be desirable to manufacture a tampon 24 having ribs, grooves and indentations. FIG. 3C provides an illustration of a tampon 24 having ribs 34, grooves 32 and indentations 400. In various embodiments, it may be desirable to manufacture a tampon 24 having ribs 34, grooves 32, indentations 400, and a raised ring 402. FIG. 3D provides an illustration of a tampon 24 having ribs 34, grooves 32, indentations 400, and two raised rings 402. In various embodiments, a press unit can be utilized to provide ribs, grooves, indentations, and/or raised rings to a tampon. While the following disclosure regarding, for example, ribs, grooves, indentations, and raised rings is provided in relation to a non-linear direction press unit, it is to be understood that other press units, such as, for example, the axial direction press units previously described and a press unit having a decreasing compression surface area which will be described later, can also provide such ribs, grooves, indentations, and/or a raised ring utilizing the disclosure as provided in relation to a non-linear direction press unit and applying it towards an axial direction press unit or a press unit which has a compression surface area which decreases with the movement of compression.

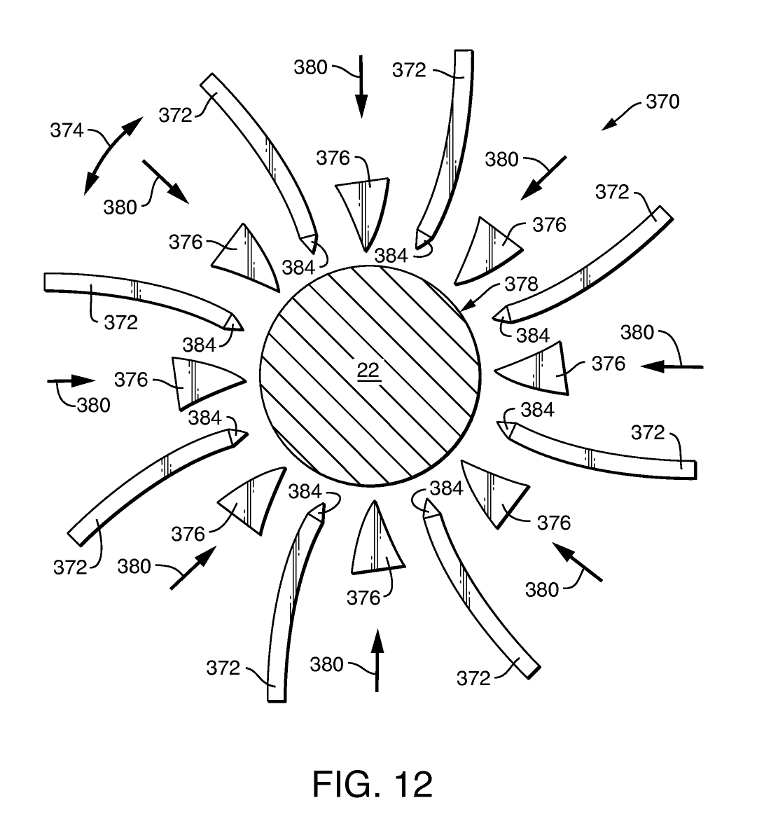

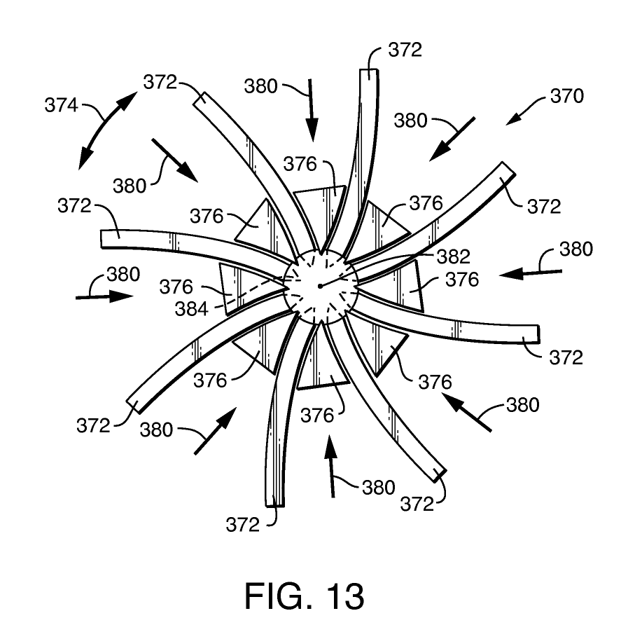

Referring to FIGS. 12 and 13, schematic illustrations of the end view of a non-linear direction press unit 370 which can provide grooves 32 and indentations 400 are illustrated. In general, the non-linear direction press unit 370 may utilize one or more dies which can reciprocate relative to one another so as to form a mold cavity 378 there between. When a material, such as a pledget 22, is positioned within the mold cavity 378, the dies may be actuated so as to move towards one another and compress the material.

Referring now to FIG. 12, an end view of an exemplary pledget 22 is illustrated in an exemplary non-linear direction press unit 370. The non-linear direction press unit 370 may include any suitable number of indentation press jaws 372. For example, the non-linear direction press unit 370 may include 1, 2, 3, 4, 5, 6, 7, 8, 9, or at least 10 indentation press jaws 372. In the embodiment of FIG. 12, eight indentation press jaws 372 are illustrated evenly spaced in the circumferential direction 374 of the pledget 22. In various embodiments, the non-linear direction press unit 370 may also include any suitable number of groove press jaws 372. For example, the non-linear direction press unit 370 may include 1, 2, 3, 4, 5, 6, 7, 8, 9, or at least 10 groove press jaws 376. The indentation press jaws 372 and the groove press jaws 376 (if present) collectively define a mold cavity 378. In the embodiment of FIG. 12, eight groove press jaws 376 are illustrated evenly spaced in the circumferential direction 374 of the pledget 22. Additionally, FIG. 12 representatively illustrates the eight indentation press jaws 372 alternately and evenly spaced with the eight groove press jaws 376 in the circumferential direction 374 of the pledget 22. Collectively, the eight indentation press jaws 372 and the eight groove press jaws 376 define the mold cavity 378.

FIG. 12 representatively illustrates the pledget 22 provided to the mold cavity 378 of the non-linear direction press unit 370 in an uncompressed configuration. Referring to FIG. 13, the non-linear direction press unit 370 of FIG. 12 is illustrated at the peak of compression in the perpendicular direction 380 (i.e., a compressed configuration). In FIG. 13, the eight indentation press jaws 372 and the eight groove press jaws 376 have moved in the direction 380 that is perpendicular to and/or radially inward towards the longitudinal centerline 382 to compress the pledget 22. The indentation press jaws 372 include one or more discrete projections 384. The discrete projections 384 penetrate the pledget 22 during the compression step to form discrete indentations 400.

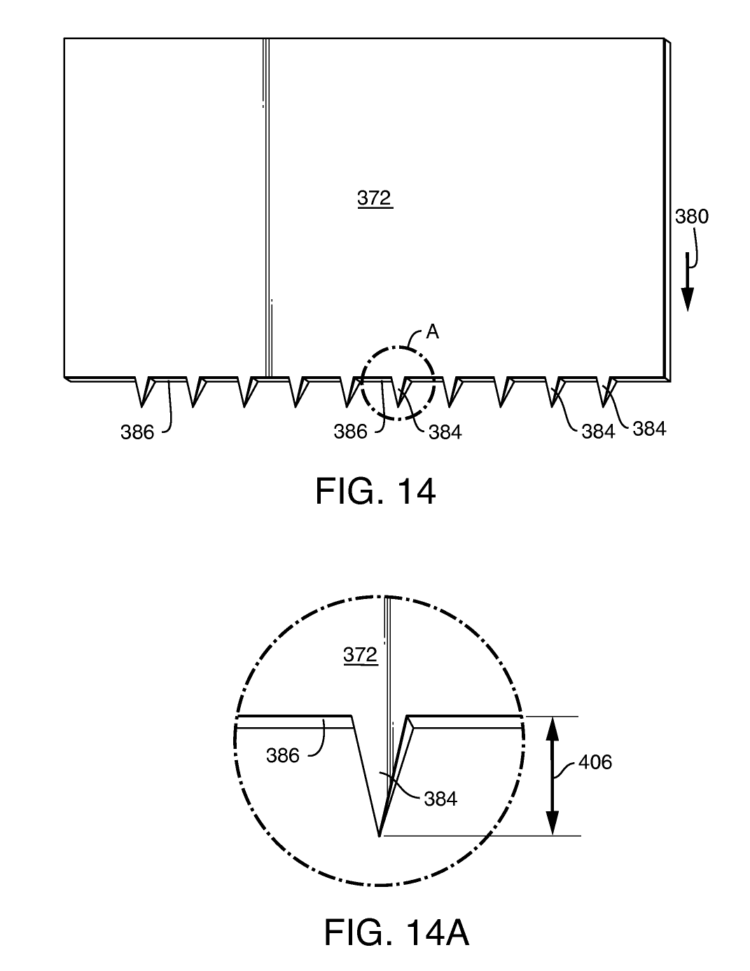

FIGS. 14, 14A, 15, 15A, 16, 16A, 17, 17A, 17B, 18 and 18A illustrate various broad side views of exemplary indentation press jaws 372 having profiling surfaces 386 and discrete projections 384 extending therefrom. The profiling surfaces 386 are adapted to compress the pledget 22 and provide shape to a portion of the outer surface of the resultant tampon 24. Likewise, the discrete projections 384 are adapted to compress the pledget 22 and then penetrate the pledget 22 to form the discrete indentations 400 that are believed to integrate the absorbent layers or structure proximate the point of penetration. The point of penetration results in an indentation 400.

In various embodiments, the discrete projections 384 can have any suitable shape, dimensions, and/or volume. In various embodiments, the discrete projections 384 can be in the shape of a pyramid, a cone, a cylinder, a cube, an obelisk, or the like, or any combination thereof. The discrete projections 384 can have a cross section that is bulbous, rectilinear, trapezoidal, polygonal, triangular, any other suitable shape, or any combination thereof. The discrete projections 384 can be in the form of a pin that is one of cylindrical, conical, elliptical, and any other suitable shape. The discrete projections 384 need not be circumferentially symmetric. The discrete projections 384 can be elongate and extend partially or entirely across the area of the profiling surface 386. The discrete projections 384 can be in wavelike formation extending partially or entirely across the area of the profiling surface 386. In various embodiments, the discrete projections 384 can have an orientation with respect to the longitudinal axis 30 of a resultant tampon 24 that is generally parallel, perpendicular, angled, or a combination of these. In various embodiments, the discrete projections 384 can be a cavity in the profiling surface 386 or a curvilinear surface on the profiling surface 386.

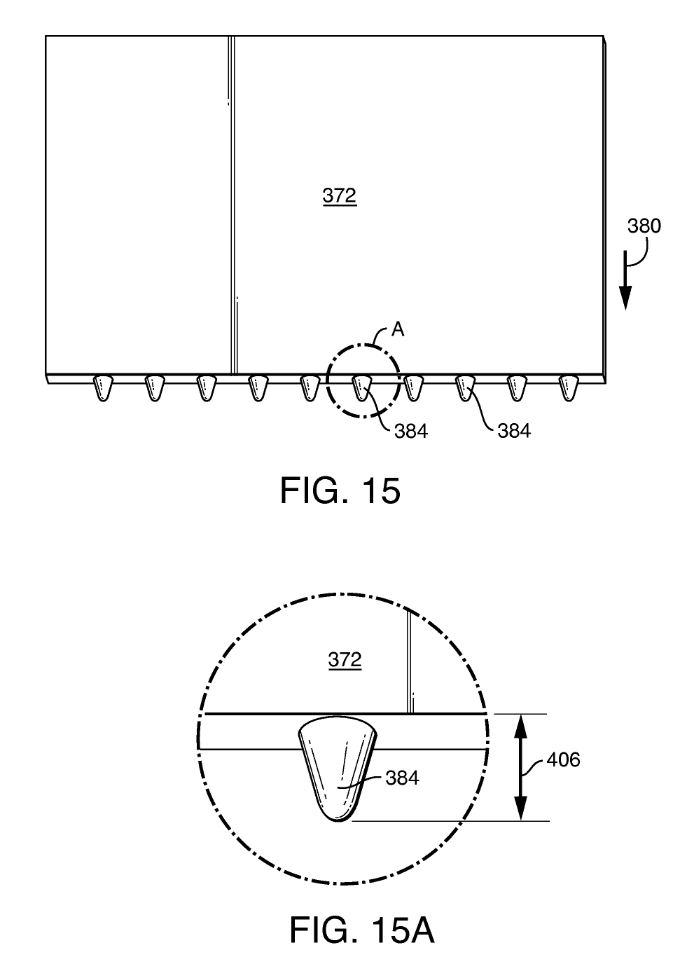

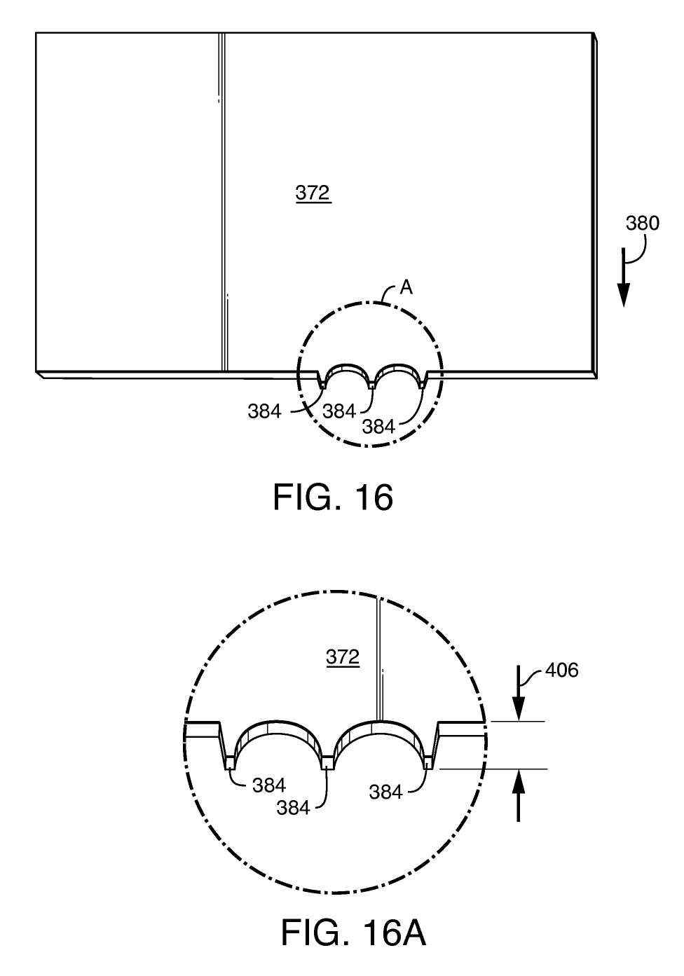

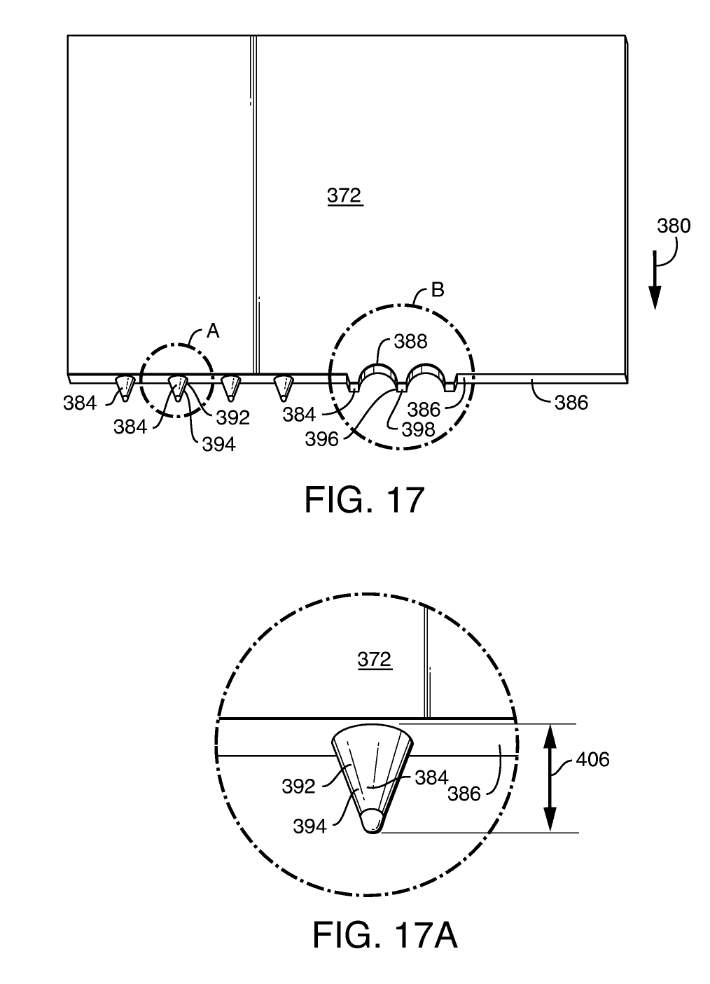

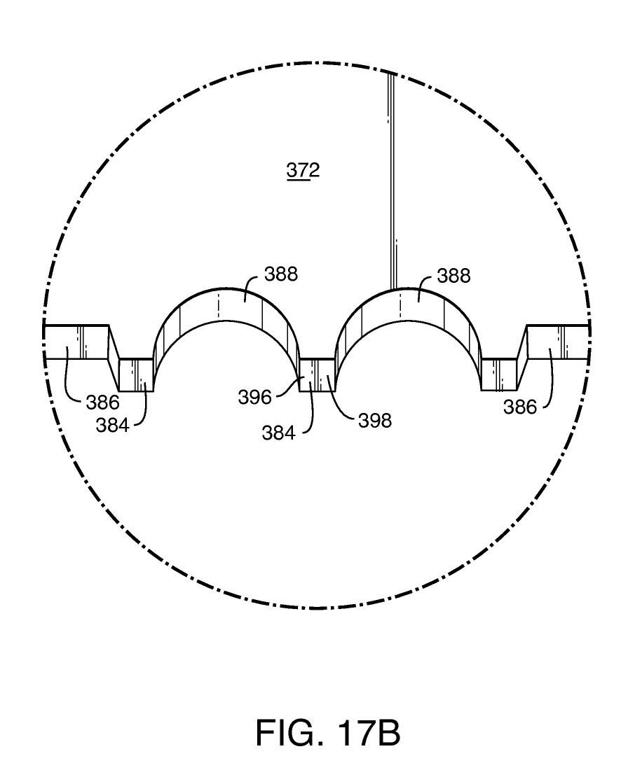

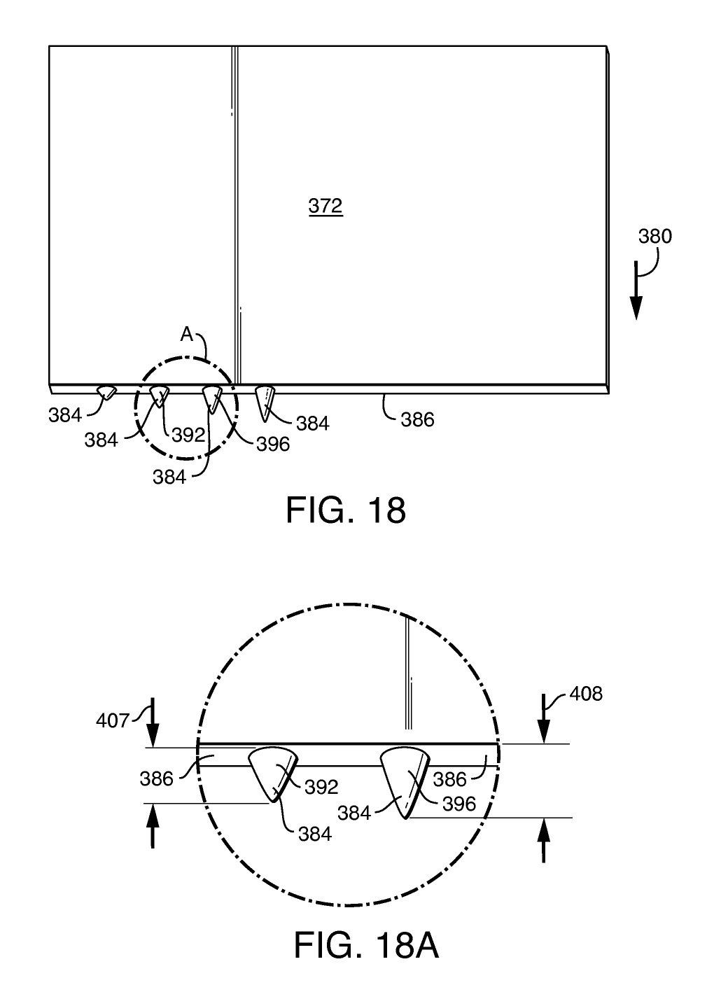

In various embodiments, the discrete projections 384 can be in the shape of a pyramid such as those illustrated in FIGS. 14 and 14A. In various embodiments, the discrete projections 384 can be in the shape of a cone with a rounded apex such as that illustrated in FIGS. 15 and 15A. In various embodiments, the discrete projections 384 can have a rectangular shape at the apex with at least one curving side such as those illustrated in FIGS. 16, 16A, 17 and 17B. In various embodiments, the discrete projections 384 can be in the shape of a cone with a relatively pointed apex such as that illustrated in FIGS. 18 and 18A.

In various embodiments, the indentation press jaws 372 can have discrete projections 384 in the form of a discrete relief 388 such as those illustrated in FIGS. 17 and 17B. The discrete relief 388 can extend into the indentation press jaw 372 and can have any suitable shape. For example, as illustrated in FIG. 17, the discrete relief 388 can have an arched shape. In such embodiments, when a plurality of indentation press jaws 372 compress the pledget 22 into the tampon 24, a circumferentially raised ring 402 is formed as illustrated in FIG. 3D.

In various embodiments, one or more of the indentation press jaws 372 can include a first discrete projection 392 having a first shape 394 and a second discrete projection 396 having a second shape 398 that is different than the first shape 394. For example, FIG. 17 representatively illustrates a first discrete projection 392 having a first shape 394 wherein the first shape 394 is a cone (FIG. 17A). FIG. 17 also representatively illustrates a second discrete projection 396 having a second shape 398, wherein the second shape 398 is more cubic.

In various embodiments, a non-linear direction press unit 370 can include a first indentation press jaw 372 having a first discrete projection 392 having a first shape 394, and a second indentation press jaw 372 having a second discrete projection 396 having a second shape 398. In various embodiments, the first shape 394 and the second shape 398 can be the same or can be different. For example, in various embodiments, the first indentation press jaw 372 can include first discrete projections 392 having the shape of cones and the second indentation press jaw 372 can include second discrete projections 396 having the shape of pyramids.

In various embodiments, the discrete projections 384 can extend any suitable distance from the profiling surface 386. For example, referring now to FIGS. 14A, 15A, 16A, and 17A, the discrete projections 384 can have an extension dimension 406 of at least 0.5, 1, 1.5, 2, 2.5, or 3 mm. In various embodiments, one or more indentation press jaws 372 can have discrete projections 384 wherein two or more of the discrete projections 384 have the same extension dimension 406 such as those illustrated in FIGS. 14 and 15. In various embodiments, one or more indentation press jaws 372 can have two or more discrete projections 384 having different extension dimensions 406 such as those illustrated in FIG. 18. FIG. 18 illustrates an indentation press jaw 372 having a profiling surface 386 wherein a first discrete projection 384 has a first extension dimension 407 (FIG. 18A) and a second discrete projection 384 has a second extension dimension 408 (FIG. 18A). As illustrated, the second extension dimension 408 is greater than the first extension dimension 407.

In various embodiments, a non-linear direction press unit 370 can include a first indentation press jaw 372 having a first discrete projection 392 having a first extension dimension 407. Likewise, the non-linear direction press unit 370 can include a second indention press jaw 372 having a second discrete projection 396 having a second extension dimension 408. In various embodiments, the first extension dimension 407 and the second extension dimension 408 can be the same or can be different. For example, in various embodiments, the first indentation press jaw 372 can include discrete projections 384 having an extension dimension 406 that is less than the extension dimension 406 of the discrete projections 384 of the second indentation press jaw 372.

Because the profiling surfaces 386 of the indentation press jaws 372 define the compressed diameter of the tampon 24, the extension dimension 406 equals the penetration depth of the discrete projection 384 into the pledget 22 during compression. The penetration depth can be defined as a percentage of the compressed diameter of the resultant tampon 24. For example, in various embodiments, the discrete projections 384 can have a penetration depth of at least about 20%, 30%, 40%, or 50% of the compressed diameter of the tampon 24. For example, in other embodiments, the compressed diameter can be about 6.6 mm and the extension dimension 406 can be about 2.55 mm such that the penetration depth is 39% of the compressed diameter.

In various embodiments, the discrete projections 384 can have a volume of at least about 3, 4, or 5 cubic millimeters. In specific embodiments, the discrete projections 384 can be blunted cones having a base diameter of about 2.523 mm and a height of about 2.546 mm for a volume of about 5.045 cubic millimeters. In various embodiments, the volume and/or the shape of the discrete projections 384 can be selected to provide the desired layer integration. In various aspects, at least about 80%, 90%, 95%, or 100% of the volume of the discrete projections 384 can penetrate the compressed tampon 24. Thus, in these embodiments, the displaced volume of absorbent material that initially forms the discrete indentations 400 is at least about 80%, 90%, 95%, or 100% of the volume of the discrete projections 384.

The tampon 24 can have a first half having an insertion end 26 and a second half having a withdrawal end 28. In various embodiments, the pledget 22 can be penetrated with discrete projections 384 in such a manner such that there are more discrete indentations 400 formed in the first half than in the second half of the resultant tampon 24. This is believed to be beneficial because the withdrawal element 14 is frequently anchored in the first half of the tampon 24 while extending from the withdrawal end 28 of the second half. As such, the withdrawal forces applied are first directed at the first half. Thus, creating greater layer integration via the discrete indentations 400 in the first half is believed to counteract the withdrawal forces and help maintain the integrity of the tampon 24. In various embodiments, the first half has at least 25%, 50%, or 75% more discrete indentations 400 than the second half. In various embodiments, all the discrete indentations 400 can be in the first half. In various embodiments, at least 60%, 70%, 80%, or 90% of the discrete indentations 400 can be in the first half.

In various embodiments, one or more raised circumferential rings 402 can be formed around the tampon 24 as illustrated in FIG. 3D. In various embodiments, a second circumferentially raised ring 402 can be formed around the tampon 24 such as illustrated in FIG. 3D. In various embodiments, the first circumferentially raised ring 402 and the second circumferentially raised ring 402 may be separated by a circumferential groove 404.

In various embodiments, the resultant tampon 24 can have one or more longitudinal rows of discrete indentations 400. In various embodiments, a first row of discrete indentations 400 can be aligned in the circumferential direction with a second row of discrete indentations 400. In various embodiments, a first row of discrete indentations 400 can be staggered in the circumferential direction with a second row of discrete indentations 400. In various embodiments, the first and second rows of discrete indentations 400 can be adjacent rows. In various embodiments, the longitudinal rows of discrete indentations 400 can extend around the circumferential direction of the tampon 24 and can be staggered such that adjacent rows of discrete indentations 400 are not aligned.

In various embodiments, one or more grooves 32 can be formed in the tampon 24. Likewise, a plurality of grooves 32 and providing a plurality of rows of discrete indentations 400 wherein the grooves 32 and the rows of discrete indentations 400 are alternated in the circumferential direction of the tampon 24 can be formed. The grooves 32 can be linear, non-linear, helical, continuous, discontinuous, wide, narrow, any other suitable shape, size, orientation, or any combination of these.

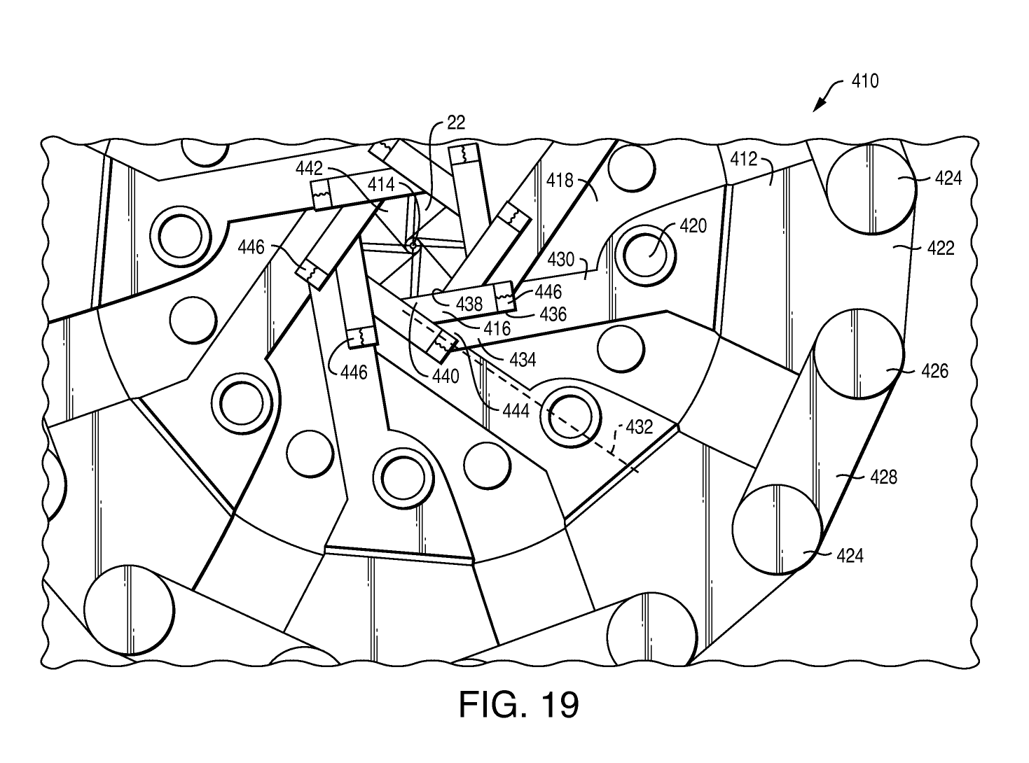

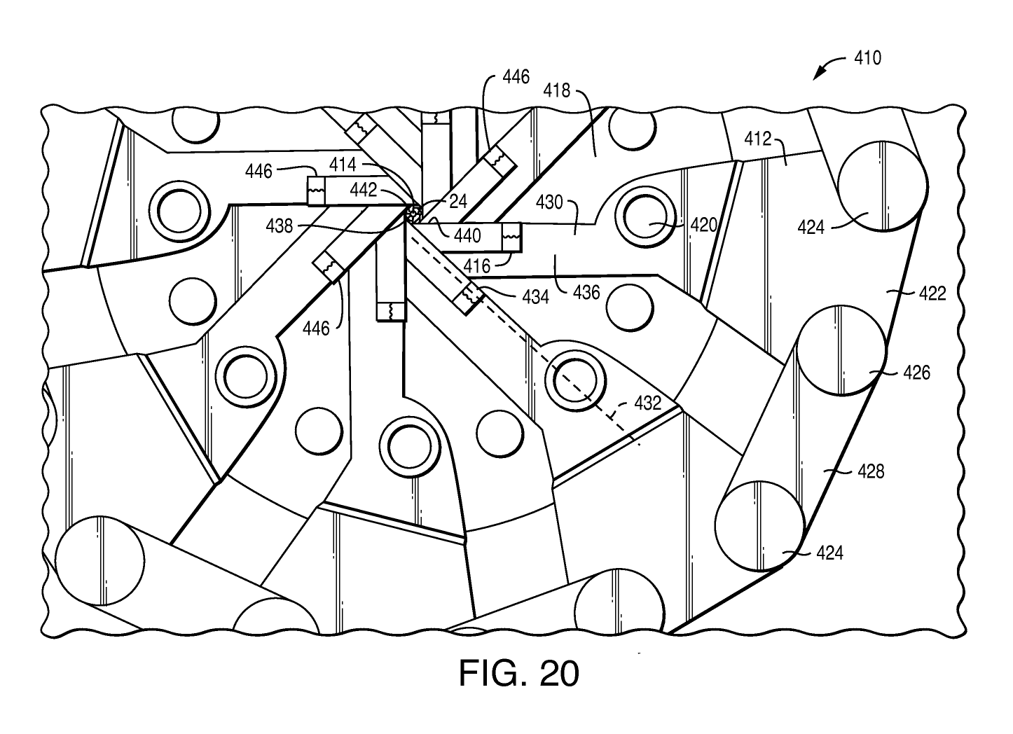

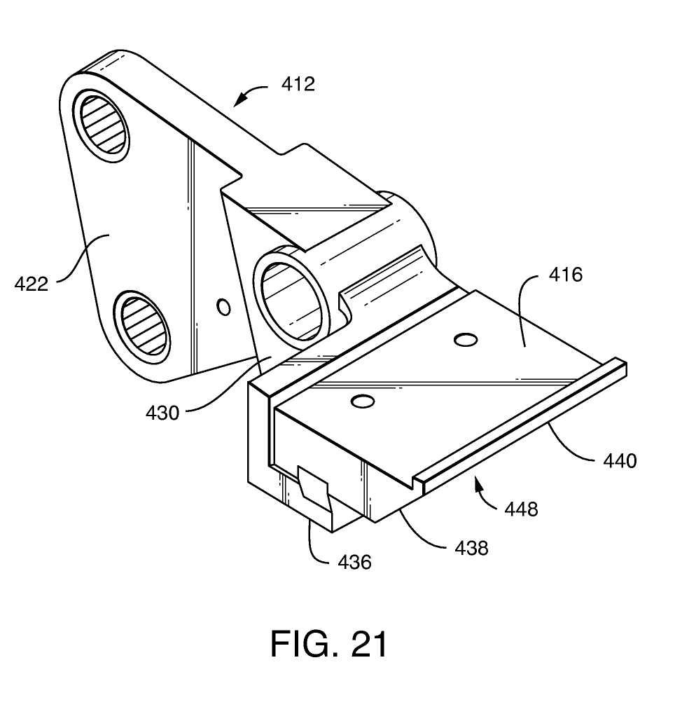

Referring to FIGS. 19 and 20, a schematic illustration of an exemplary embodiment of a press unit 410 which can have a compression surface area which decreases during the movement of compression is illustrated. The press unit 410 can have compressing surfaces and a compressing mechanism to move the compressing surfaces in a non-linear motion while compressing the material. As the press unit 410 compresses, the compressing surface area decreases and circumferential gapping is maintained close to zero over the relevant range of the press unit 410. The operating range of the press unit 410 is defined as the range between the maximum compression diameter and the minimum compression diameter. The ratio of the initial compression diameter to the final compression diameter, or the compression ratio, obtainable with this press unit 410 is greater than 1, 2, 3, 4, 5, 6, 7, 8, 9, 10, 15, or 20. The initial compression diameter is the effective diameter of the material prior to compression, which is essentially the minimum diameter to which the press unit 410 must be opened to accept the material. The diameter in the preceding terms is the diameter of the hypothetical cylinder 442 defined below. The final compression diameter is the desired diameter of the material after compression. By maintaining circumferential gapping close to zero over the relevant range of the press unit 410, the compression jaws can reinforce each other to improve apparatus stability.

A press unit 410 for manufacturing an exemplary tampon 24 is illustrated in FIGS. 19 and 20. The press unit 410 used as an example here includes eight levers 412 (see FIGS. 19-21), although any suitable number of levers 412 can be accommodated. The center of the press unit 410 defines a central longitudinal axis 414, which is the point at which the jaws 416 meet when the levers 412 and jaws 416 are at their innermost extent of travel. Each lever 412 is connected to a fixed ring 418 with a pivot pin 420 and is pivotable within certain limits about the pivot pin 420. Each lever 412 has a lever outer end 422 that is pivotably linked by first and second coupling pins 424, 426 to adjacent chain links 428 as a part of a drive mechanism (not shown). The first and second coupling pins 424, 426 and the pivot pins 420 can each be positioned in generally circular array, or in any other suitable array. The spacing between adjacent coupling pins 424, 426 and between adjacent pivot pins 420 is determined by the number of levers 412 to be included within the circle.

The levers 412 are designed as angle levers and each includes a lever arm 430 that is positioned radially inwardly. Each lever 412 has a lever longitudinal axis 432 extending from the lever outer end 422 through the pivot pin 420 to a radially-inward end portion 434 of each lever arm 430. The radially-inward end portion 434 includes a jaw 416 used in compression. The jaw 416 can be formed integrally with the lever arm 430 and therefore be a portion of the lever 412 itself, the jaw 416 can be attached to the lever arm 430 at a tool carrier 436 on the radially-inward end portion 434 of the lever arm 430, or the jaw 416 can be associated with the lever 412 in any suitable manner. In various embodiments, the number of levers 412 and jaws 416 can be 3, 4, 5, 6, 8, 10, 12, 16, or any other suitable number.

Each jaw 416 includes a compression surface 438 and a jaw edge 440. The compression surface 438 defines a plane that is generally parallel to the lever longitudinal axis 432. Each jaw 416 projects toward an adjacent jaw 416 where the adjacent jaw 416 is positioned in a clockwise direction from the first jaw 416. The jaw edge 440 of one jaw 416 is disposed in the vicinity of the compression surface 438 of the clockwise-adjacent jaw 416. The topography of a given jaw edge 440 essentially matches the topography of the compression surface 438 of an adjacent jaw 416. The press unit 410 is arranged such that a plane defined by the compression surface 438 of each jaw 416 is at all points in the compression cycle tangential to the central longitudinal axis 414.

In addition, each compression surface 438 defines an area that is exposed to the material to be compressed. This area is generally between the jaw edge 440 of a particular jaw 416 and a line or point projected on that jaw 416 by the plane of the compression surface 438 of an adjacent jaw 416, or that is contacted by or adjacent to the jaw edge 440 of an adjacent jaw 416. For example, a press unit 410 with eight jaws 416 cooperate to form a generally octagonal compression cavity. One side of that octagon defines the area of a compression surface 438 exposed to the material to be compressed. As the jaws 416 move inwardly, the octagon shrinks, and the area of each side and therefore each compression surface 438 decreases. The compression surfaces 438 define a hypothetical cylinder 442 that is, in a radial direction, a hypothetical circle of maximum diameter that can be inscribed within the compression surfaces 438. In the example described in this paragraph, the circle is a circle of maximum diameter that is inscribed within the octagon defined by the compression surfaces 438. As a result, as the jaws 416 move inwardly, the hypothetical cylinder 442 also shrinks in diameter.

Activating the drive mechanism and rotating the chain link 428 causes the lever 412 to pivot about the pivot pin 420. The lever 412 pivots such that the radially-inward end portion 434 of the lever arm 430 moves radially inward when the chain link 428 is rotated in a clockwise direction in this example. Each compression surface 438 moves radially inwardly with the end portion 434 to which it is attached. Thus, the press unit 410 closes when the chain link 428 is rotated in a clockwise direction in this example, and the press unit 410 opens when the chain link 428 is rotated in a counterclockwise direction in this example. It can be seen that the jaws 416, and particularly a point on a jaw 416, can be configured to move in a non-linear manner, or in a curvilinear manner depending on the arrangement of levers, pins, fixed rings, and chain links.

The press unit 410 can theoretically move inwardly until the jaw edge 440 of each jaw 416 meets the others at the central longitudinal axis 414 of the press unit 410. In other words, the jaws 416 can move inwardly until the hypothetical cylinder 442 defined by the compression surfaces 438 reaches a diameter of zero.

FIG. 19 illustrates that in the open starting position the jaw edges 440 of the jaws 416 are not directed toward the central longitudinal axis 414 of the press unit 410 but tangentially toward the hypothetical cylinder 442 that surrounds the central longitudinal axis 414 at a selected distance. Thus it is achieved that the compression forces that are applied by the jaws 416 are not centrally but tangentially directed toward a circle that surrounds the material to be manufactured at a selected distance.

In the open starting position of the press unit 410 according to FIG. 19, a pledget 22 is inserted into the opening between the compression surfaces 438. By rotating the chain links 428 clockwise relative to the fixed ring 418, the compression surfaces 438 are first brought into an intermediate position and finally into the end position illustrated in FIG. 20. With this pivoting movement, the levers 412 are pivoted about the pivot pins 420. A comparison of FIG. 20 with FIG. 19 shows that during this movement the deformation forces that are applied by the compression surfaces 438 lead to a volume reduction of the pledget 22 that is uniform about the periphery and transform the pledget 22 into a tampon 24. After slightly opening the jaws, the tampon 24 is removed from the press unit 410.

The press unit 410 incorporates multiple compression jaws 416 that cooperate with each other such that the clearance between adjacent jaws 416 defines a gap 444 at some points in the compression cycle. The gap 444 defines a gap centerline, which connects the series of midpoints of the gap between adjacent jaws 416. A line including the gap centerline of the gap 444 between a first jaw 416 and an adjacent second jaw 416 is sometimes parallel to the compression surface 438 of the adjacent second jaw 416. As a result, a line including the gap centerline will generally be parallel to a tangent to the hypothetical cylinder 442, and will not intersect the central longitudinal axis 414. In the press unit 410, the orientation of the gaps 444 helps prevent intrusion of material into the gap 444. In other words, the gap 444 between adjacent jaws 416 provides a substantially reduced clearance profile in the direction of compression between adjacent jaws 416 during the entire compression cycle, thereby substantially reducing the gaps 444 in which material can be captured. In addition, geometric analysis of the structure of the press unit 410 shows that the gap 444 changes over the compression cycle and is minimized at both minimum and maximum compression diameters. In one aspect the substantially-reduced clearance between adjacent jaws 416 approaches zero such that there is no practical gap 444 present at minimum compression, such that migration of material around the contacting surfaces is substantially limited.

The attachment of the jaw 416 to the tool carrier 436 can include a biasing mechanism 446 configured to urge the jaw 416 in a direction away from the pivot pin 420 and toward a clockwise-adjacent jaw 416. In other words, the biasing mechanism 446 pushes the jaw 416 toward a clockwise-adjacent jaw 416, whereas such clockwise-adjacent jaw 416 resists such pushing. In this manner, any gap that would otherwise exist between adjacent jaws 416 will be closed by the contact between adjacent jaws 416.

The biasing mechanism 446 can be any suitable mechanism, component, force, or combination of these capable of biasing a jaw 416 toward an adjacent jaw 416. The biasing mechanism 446 can be disposed on one or more of a lever 412, jaw 416, and any other element of the press unit 410. The biasing mechanism 446 can be disposed between a lever 412 and a jaw 416, particularly on, in, or in the vicinity of a tool carrier 436. Suitable biasing mechanisms 446 include, but are not limited to, bevel, tension, and compression springs; pneumatic and/or hydraulic components including cylinders or bladders; elastomeric components such as an elastomeric block or an elastomeric band; mechanical gearing such as a rack and pinion or non-circular gearing; a cam mechanism including followers or a contoured wedge mechanism; electrical components including a solenoid; magnetic forces; vacuum; mechanical engagement such as a t-slot pin-type mechanism; a supplemental linkage connected between two or more jaws 416, and any combination of these. The biasing mechanism 446 can be disposed directly on or near the jaws 416, or can be external components that direct influence to the jaws 416.

The press unit 410 can be used to make a tampon 24 having increased layer or structure integration. The addition of one or more shaping elements 448 to the press unit 410 can be used to impart indentations, grooves, bulges, and any other suitable topographical elements to the material. FIG. 21 illustrates a perspective view of a jaw 416 having a shaping element 448. As noted above, grooves, ribs, indentations and raised rings can be provided to a tampon 24 utilizing a press unit 410 having a decreasing compression surface area in a manner similar to that described for incorporating grooves, ribs, indentations, and raised rings into a tampon 24 utilizing a non-linear direction press unit. The shaping element 448 can be modified in a manner similar to the indention press jaw 372 described above.

As described herein, a press unit can provide compression in the axial direction, non-linear direction, or can have a compress surface area which decreases during the movement of compression. Also as described herein, the material can be compressed into a tampon or pessary and can be provided with various grooves, ribs, indentations, raised rings, etc. The grooves, ribs, indentations, raised rings, etc. can be provided in any pattern as deemed suitable. In various embodiments, each of the press units carried by an apparatus can produce multiple identical tampons or pessaries. In various embodiments, an apparatus can carry at least two press units which can produce at least two tampons or pessaries which are not identical.

### Method of Compression:

The apparatus disclosed herein, can be utilized in the manufacturing process of a tampon or pessary. The apparatus can be utilized to compress the pledget or the uncompressed pessary into a tampon or compressed pessary having a size and dimension more suitable for insertion into the vaginal cavity either digitally or through the use of an application.

In various embodiments, the process of using an apparatus as described herein can include providing the apparatus. The apparatus can include multiple press unit support structures rotatable about an axis and at least one press unit associated with each press unit support structure. The press units can be any of those described herein, such as, for example, an axial press unit, a non-linear direction press unit, a press unit having a compression surface area which can decrease, or a combination of the described press units. During a revolution of each press unit support structure about an axis, a material which has been loaded into one of the press units can undergo a complete compression cycle of a press unit. During the compression cycle, the press unit can transition from the full open configuration, through a partially closed configuration to a full closed configuration and from the full closed configuration, through a partially open configuration, to the full open configuration. The press unit can begin to compress the material in the partial closed configuration and the compressed material can dwell in the full closed configuration for the desired length of time during the revolution of the press nit about the axis. Following the desired length of dwell, the press unit can transition through the partially open configuration to the full open configuration.

A material, such as, for example, a pledget or an uncompressed pessary can be loaded into one of the press units carried by one of the press unit support structures. The initial positioning of the material within the press unit can be referred to as the zero degree position of the press unit support structure. During the loading of the material into the press unit, the press unit can be in a full open configuration and the material to be compressed can be loaded into the open press unit. Once the material to be compressed is loaded into the open press unit, the compression cycle can begin to transition the press unit from the full open configuration, through a partially closed configuration and to a full closed configuration. It should be understood that as the press unit transitions from a full open configuration to a full closed configuration, the press unit will transition through a partially closed configuration during which time the volume of the chamber containing the material to be compressed will become smaller in volume until the press unit reaches the full closed configuration. In other words, as the press unit is in a partially closed configuration, the material located within the press unit can begin to be compressed.

As the press unit continues to progress through the compression cycle, the press unit support structure carrying the press unit can rotate about the axis. When the press unit is in a full closed configuration, the material located within the press unit can be under full compression at the desired level of compression. The compression of the material located in a press unit can occur during the revolution of its respective press unit support structure from the zero degree position until at least about the 90, 120, 150, 180, 210, 240, 270, 300 or 330 degree position ±10°. When the material has been compressed to the desired level of compression, the press unit can begin to transition from a full closed configuration, through a partially open configuration and back to the full open configuration to allow for unloading of the material. As the press unit is transitioning through the partially open configuration, the chamber within which the material is loaded can begin to increase in volume. As described above, in some embodiments, it may be desirable to unload the material while the press unit is in a partially open configuration. Also as described above, in some embodiments, it may be desirable to unload the material when the press unit has reached the full open configuration. Following the unloading of the material, whether during the partially open configuration or the full open configuration of the press unit, the press unit can return to a full open configuration for loading of another material to begin the compression cycle.

As noted above, an apparatus can carry a plurality of individual press units on multiple press unit support structures. In an embodiment, during a revolution of each of the press unit support structures about an axis, each press unit can be operated and actuated synchronously with each other press unit carried by the other press unit support structures as the press unit support structures rotate about an axis. In other words, each press unit can be in phase with each other press unit. When the press units are in phase with each other, they can each undergo the configurations of the compression cycle in synchronicity with each other press unit. In an embodiment, during a revolution of the press unit support structures about an axis, each press unit can be operated and actuated independently of any other press unit carried by the other press unit support structures as the press unit support structures rotate about an axis. In other words, each press unit can be out of phase with each other press unit. When the press units are out of phase with each other, they can each be experiencing a different configurations of the compression cycle at any moment in time.

In various embodiments, each press unit carried by a press unit support structure can be in phase with each other press unit carried by the other press unit support structures. In such embodiments, each press unit can experience each configuration of the compression cycle at substantially the same time. For example, in a revolution of the press unit support structures about an axis, each press unit can have a material loaded into the press unit at substantially the same time during the compression cycle. Each press unit support structure can continue to rotate about an axis, and each press unit can transition from the full open configuration to the full closed configuration at substantially the same time. The press unit support structures can continue to rotate about the axis, and following the compression of the material in each press unit, the press units can transition from the full closed configuration to the full open configuration. As described above, the compressed material can be unloaded from the press units during the transition from the full closed configuration to the full open configuration, i.e., in the partially open configuration, or when the press units have reached the full open configuration. In various embodiments, at a moment in time during the revolution of the press unit support structures about an axis, at least two press units can be in a full open configuration. In various embodiments, at a moment in time during the revolution of the press unit support structures about an axis, at least two press units can be in a partially closed configuration. In various embodiments, at a moment in time during the revolution of the press unit support structures about an axis, at least two press units can be in a full closed configuration. In various embodiments, at a moment in time during the revolution of the press unit support structures about an axis, at least two press units can be in a partially open configuration.

In various embodiments, each press unit carried by a press unit support structure can be out of phase with each other press unit carried by the other press unit support structures. In such embodiments, each press unit can be experiencing a different configuration of the compression cycle at any moment in time during the revolution of each of the press unit support structures about an axis. For example, in a revolution of each of the press unit support structures about an axis, at an initial moment in time, a material can be loaded into a first press unit. The press unit support structures can continue to rotate about the axis and the first press unit can transition from the full open configuration to the full closed configuration to compress the material loaded within the first press unit. While the first press unit is undergoing the transition from the full open configuration to the full closed configuration, a second material can be loaded into a second press unit for compression. It should be understood that the second material can be loaded into the second press unit while the first press unit is in any of the configurations of a partially closed configuration, a full closed configuration, a partially open configuration or a full open configuration. As the press units can be out of phase, it can be possible, in various embodiments, to load a material for compression into one press unit at substantially the same time as a compressed material is being unloaded from another press unit. In various embodiments, at a moment in time during the revolution of the press unit support structures about an axis, at least two press units can be in a full open configuration. In various embodiments, at a moment in time during the revolution of the press unit support structures about an axis, at least two press units can be in a partially closed configuration. In various embodiments, in a moment of time during the revolution of the press unit support structures about an axis, at least two press units can be in a full closed configuration. In various embodiments, at a moment in time during the revolution of the press unit support structures about an axis, at least two press units can be in a full open configuration, a partially closed configuration, a full closed configuration, or a partially open configuration and at least one press unit can be in a full open configuration, a partially closed configuration, a full closed configuration, or a partially open configuration. In such embodiments, the two press units can be either in the same configuration as each other or can be in different configurations from each other.

In the interests of brevity and conciseness, any ranges of values set forth in this disclosure contemplate all values within the range and are to be construed as support for claims reciting any sub-ranges having endpoints which are whole number values within the specified range in question. By way of hypothetical example, a disclosure of a range of from 1 to 5 shall be considered to support claims to any of the following ranges: 1 to 5; 1 to 4; 1 to 3; 1 to 2; 2 to 5; 2 to 4; 2 to 3; 3 to 5; 3 to 4; and 4 to 5.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An apparatus characterized by comprising:
a. a first press unit support structure and a second press unit support structure, each of the first press unit support structure and the second press unit support structure rotatable about an axis;
b. an axial direction press unit associated with the first press unit support structure; and
c. a second press unit associated with the second press unit support structure wherein the second press unit is one of an axial direction press unit, a non-linear direction press unit, or a press unit having a compression surface area which decreases over time.

2. The apparatus of claim 1 wherein at a moment in time during a revolution of each of the first and second press unit support structures about the axis, the axial direction press unit is in a configuration which is one of a full open configuration, partially closed configuration, a partially open configuration, or a full closed configuration and the second press unit is in a configuration which is one of a full open configuration, a partially closed configuration, a partially open configuration, or a full closed configuration

3. The apparatus of claim 2 wherein the configuration of the axial direction press unit is the same as the configuration of the second press unit.

4. The apparatus of claim 2 wherein the configuration of the axial direction press unit is different from the configuration of the second press unit.

5. The apparatus of claim 1 wherein the axial direction press unit is associated with the first press unit support structure in a variable spatial relationship relative to second press unit which is associated with the second press unit support structure.

6. The apparatus of claim 1 wherein compression of a material within one of the axial press unit or second press unit begins after the axial press unit or second press unit rotates from a zero degree position and continues to a rotation of at least about a 90 degree position.

7. An apparatus characterized by comprising:
a. a first press unit support structure and a second press unit support structure, each of the first press unit support structure and the second press unit support structure rotatable about an axis;
b. a non-linear direction press unit associated with the first press unit support structure; and
c. a second press unit associated with the second press unit support structure wherein the press unit is one of an axial direction press unit, a non-linear direction press unit, or a press unit having a compression surface area which decreases with the movement of compression.

8. The apparatus of claim 7 wherein at a moment in time during a revolution of each of the first and second press unit support structures about the axis, the non-linear direction press unit is in a configuration which is one of a full open configuration, a partially closed configuration, a full closed configuration or a partially open configuration and the second press unit is in a configuration which is one of a full open configuration, a partially closed configuration, a full closed configuration or a partially open configuration.

9. The apparatus of claim 8 wherein the configuration of the non-linear direction press unit is the same as the configuration of the second press unit.

10. The apparatus of claim 8 wherein the configuration of the non-linear direction press unit is different from the configuration of the second press unit.

11. The apparatus of claim 7 wherein the non-linear direction press unit is associated with the first press unit support structure in a variable spatial relationship relative to second press unit which is associated with the second press unit support structure

12. The apparatus of claim 7 wherein compression of a material within one of the non-linear direction press unit or the second press unit begins after the non-linear direction press unit or the second press unit rotates from a zero degree position and continues to a rotation of at least about a 90 degree position.

13. An apparatus characterized by comprising:
a. a first press unit support structure and a second press unit support structure, each of the first press unit support structure and the second press unit support structure rotatable about an axis;
b. a first press unit having a compression surface area which decreases with the movement of compression associated with the first press unit support structure; and
c. a second press unit associated with the second press unit support structure, wherein the second press unit is one of an axial direction press unit, a non-linear direction press unit, or a press unit having a compression surface area which decreases with the movement of compression.

14. The apparatus of claim 13 wherein at a moment in time during a revolution of each of the first and second press unit support structures about the axis, the first press unit is in a configuration which is one of a full open configuration, a partially closed configuration, a partially open configuration, or a full closed configuration and the second press unit is in a phase which is one of a full open configuration, a partially closed configuration, a partially open configuration or a full closed configuration.

15. The apparatus of claim 14 wherein the configuration of the first press unit is the same as the configuration of the second press unit.

16. The apparatus of claim 14 wherein the configuration of the first press unit is different from the configuration of the second press unit.

17. The apparatus of claim 13 wherein the first press unit is associated with the first press unit support structure in a variable spatial relationship relative to the second press unit which is associated with the second press unit support structure.

18. The apparatus of claim 13 wherein compression of a material within one of the first or second press units begins after the first or second press units rotates from a zero degree position and continues to a rotation of at least about a 90 degree position.
